(19) United States
(12) Patent Application Publication
(10) Pub. No.: US 2021/0032358 A1
(43) Pub. Date: Feb. 4, 2021

(54) PHARMACEUTICAL COMPOSITIONS COMPRISING BISPECIFIC ANTIBODIES DIRECTED AGAINST CD3 AND CD20 AND THEIR USES

(71) Applicant: GENMAB A/S, Copenhagen V (DK)

(72) Inventors: Jesper VALBJOERN, Copenhagen (DK); Lene Schantz HARLOW, Copenhagen (DK); Jacob Dahlqvist CLAUSEN, Copenhagen (DK); Mette Hamborg JENSEN, Copenhagen (DK); Christian CIMANDER, Copenhagen (DK); Peter Juul MADSEN, Holbæk (DK)

(21) Appl. No.: 16/967,365

(22) PCT Filed: Feb. 8, 2019
(86) PCT No.: PCT/EP2019/053178

§ 371 Date: Aug. 4, 2020

(30) Foreign Application Priority Data
Feb. 9, 2018 (EP) 18156050.9

(51) Int. Cl. C07K 16/28; A61K 9/00; A61K 47/12; A61K 47/26

(52) U.S. Cl. CPC C07K 16/2887; C07K 16/2809; A61K 9/0019; C07K 2317/31; A61K 47/26; C07K 2317/565; C07K 2317/52; A61K 47/12

(57) ABSTRACT

The present invention relates to pharmaceutical compositions and dosage unit forms of bispecific CD3xCD20 antibodies and to routes of administration.

### FIELD OF THE INVENTION

The present invention relates to pharmaceutical compositions and unit dosage forms of bispecific antibodies directed against CD3 and CD20 and their uses.

### BACKGROUND OF THE INVENTION

CD3 has been known for many years and therefore has been subject of interest in many aspects. Specifically antibodies raised against CD3 or the T-cell Receptor Complex, which CD3 is part of, are known.

A promising approach to improve targeted antibody therapy is by delivering cytotoxic cells specifically to the antigen-expressing cancer cells. This concept of using T-cells for efficient killing of tumor cells has been described in Staerz, et. al., 1985, Nature 314:628-631). However, initial clinical studies were rather disappointing mainly due to low efficacy, severe adverse effects (cytokine storm) and immunogenicity of the bispecific antibodies (Muller and Kontermann, 2010, BioDrugs 24: 89-98). Advances in the design and application of bispecific antibodies have partially overcome the initial barrier of cytokine storm and improved clinical effectiveness without dose-limiting toxicities (Garber, 2014, Nat. Rev. Drug Discov. 13: 799-801; Lum and Thakur, 2011, BioDrugs 25: 365-379). Critical to overcome the initial barrier of cytokine storm as described for catumaxomab (Berek et al. 2014, Int. J. Gynecol. Cancer 24(9): 1583-1589; Mau-Sorensen et al. 2015, Cancer Chemother. Pharmacol. 75: 1065-1073), was the absence or silencing of the Fc domain.

The CD20 molecule (also called human B-lymphocyte-restricted differentiation antigen or Bp35) is a hydrophobic transmembrane protein with a molecular weight of approximately 35 kD located on pre-B and mature B lymphocytes (Valentine et al. (1989) J. Biol. Chem. 264(19):11282-11287; and Einfield et al., (1988) EMBO J. 7(3):711-717). CD20 is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin's lymphomas (NHL) (Anderson et al. (1984) Blood 63(6):1424-1433), but is not found on hematopoietic stem cells, pro-B cells, normal plasma cells, or other normal tissues (Tedder et al. (1985) J. Immunol. 135(2):973-979).

Methods for treating cancer as well as autoimmune and immune diseases by targeting CD20 are known in the art. For example, the chimeric CD20 antibody rituximab has been used for or suggested for use in treating cancers such as non-Hodgkin's lymphoma (NHL), chronic lymphocytic leukemia (CLL) and small lymphocytic lymphoma (SLL). The human monoclonal CD20 antibody ofatumumab has been used for or suggested for use in treating among others various CLL indications, follicular lymphoma (FL), neuromyelitis optica (NMO), diffuse and relapsing-remitting multiple sclerosis (RRMS).

Bispecific antibodies that bind to both CD3 and CD20 are known from the prior art. WO2011028952 describes amongst others the generation of CD3xCD20 bispecific molecules using Xencor's XmAb bispecific Fc domain technology.

WO2014047231 describes REGN1979 and other CD3xCD20 bispecific antibodies generated using the FcΔAdp technology from Regeneron Pharmaceuticals.

Sun et al. (2015, Science Translational Medicine 7, 287ra70) describe a B cell-targeting anti-CD20/CD3 T cell-dependent bispecific antibody constructed using “knobs-into-holes” technology.

WO 2016/110576, incorporated herein by reference, provides bispecific CD3xCD20 antibodies and the present invention relates to stable pharmaceutical formulations of the CD3xCD20 antibodies of WO 2016/110576. Bispecific antibodies that bind to both CD3 and CD20 may be useful in therapeutic settings in which specific targeting and T cell-mediated killing of cells that express CD20 is desired, and such bispecific antibodies are being investigated for the potential treatment of NHL, CLL, and other and other B-cell malignancies. Prior art CD3xCD20 bispecific antibodies which are under development in clinical trials are being administered via the intravenous (IV) route. Such administration route may lead to a high Cₘₐₓ for the CD3xCD20 bispecific antibody which may be associated with too high levels of cytokine release; Cross-linking of the target cell expressing CD20 and a T cell by bispecific antibodies leads to the release of cytokines, for example to the release of proinflammatory cytokines, e.g. IL-6, TNF-alpha or IL-8, resulting in adverse effects like fever, nausea, vomiting and chills. Thus, despite the unique anti-tumor activity of bispecific antibodies, their immunological mode of action triggers unwanted “side” effects, i.e. in the induction of unwanted inflammatory reactions known e.g. as “first dose cytokine response or syndrome”. Under such circumstances, patients need to be subjected to a concomitant treatment or premedication with e.g. analgesics, antipyretics, and/or nonsteroidal anti-inflammatory drugs. Thus, there is an unmet need for modifying or reducing the systemic cytokine release profile of T-cell redirecting bispecific antibodies upon their administration to humans or animals. Therefore, there is a need for additional antibody formulations and pharmaceutical compositions of bispecific antibodies binding CD3 and CD20, which compositions can be administered differently to avoid or to reduce the side effects of systemic cytokine release but at the same time provide highly efficient T cell-mediated killing of tumor cells that express CD20. It is an object of the present invention to provide a simple and stable pharmaceutical formulation of the CD3xCD20 bispecific antibodies disclosed in WO 2016/110576 where the bispecific CD3xCD20 antibody and the formulation as such is stable over a broad range of antibody concentrations even at high antibody concentrations of about 60 mg/mL or at 120 mg/mL or even 150 mg/mL. It is a further object of the present invention to provide a pharmaceutical formulation of the CD3xCD20 bispecific antibodies which formulation is stable over a period of at least 3 months, or even longer, such as at least 6 month or at least 12 months. Further it is an object of the present invention to provide a formulation which is stable over a range of temperatures such as from 2° to 25° C. It is a further object to provide a pharmaceutical formulation of a bispecific CD3xCD20 antibody which is suitable both for IV and subcutaneous administration. In many cases it may be more convenient for patients that the pharmaceutical formulation is administered subcutaneously as the infusion/injection time is much shorter for SC administration compared to IV administration. It is a further object of the present invention to provide a pharmaceutical formulation of a bispecific CD3xCD20 antibody which is well tolerated at the SC injection site. It is a further object to provide a pharmaceutical composition which may be administered subcutaneously to give reduced cytokine release profile in patients but at the same time same time provide highly efficient T cell-mediated killing of tumor cells that express CD20.

### SUMMARY OF THE INVENTION

It is an object of the present invention to provide novel pharmaceutical compositions of bispecific antibodies comprising a first antigen-binding region derived from a CD3 antibody and a second antigen-binding region derived from a CD20 antibody.

The novel compositions comprising CD3xCD20 bispecific antibodies are useful in therapeutic settings in which specific targeting and T cell-mediated killing of cells that express CD20 is desired. The formulations are useful both for IV administration and for subcutaneous administration.

Accordingly, in a main aspect the present invention relates to a pharmaceutical composition comprising:
a. 50 to 120 mg/mL of a bispecific antibody binding to human CD3 and human CD20,
b. 20 to 40 mM acetate,
c. 140 to 160 mM sorbitol,
where the pH of the composition is between 5 and 6 and where the bispecific antibody comprises a first binding region binding to human CD3 which comprises the CDR sequences:
   VH-CDR1: SEQ ID NO: 1
   VH-CDR2: SEQ ID NO:2
   VH-CDR3: SEQ ID NO: 3
   VL-CDR1: SEQ ID NO: 4
   VL-CDR2: GTN, and
   VL-CDR3: SEQ ID NO: 5
and a second binding region binding to human CD20 which comprises the CDR sequences:
   VH-CDR1: SEQ ID NO: 8
   VH-CDR2: SEQ ID NO: 9
   VH-CDR3: SEQ ID NO: 10
   VL-CDR1: SEQ ID NO: 11
   VL-CDR2: DAS, and
   VL-CDR3: SEQ ID NO: 12.

In a further aspect, the present invention relates to the use of the pharmaceutical composition of the invention for subcutaneous administration.

In a further aspect, the present invention relates to the use of the pharmaceutical composition of the invention for intravenous administration.

In a further aspect, the present invention relates to the use of the pharmaceutical composition of the invention for the treatment of cancer.

In a further aspect, the present invention relates to a method of treating cancer in a subject comprising administering to a subject in need thereof the pharmaceutical composition of the invention for a time sufficient to treat the cancer.

In yet a further aspect the invention relates to a unit dosage form, comprising
a. a bispecific antibody comprising a first binding region binding to human CD3 which comprises the CDR sequences:
   VH-CDR1: SEQ ID NO:1
   VH-CDR2: SEQ ID NO: 2
   VH-CDR3: SEQ ID NO: 3
   VL-CDR1: SEQ ID NO: 4
   VL-CDR2: GTN, and
   VL-CDR3: SEQ ID NO: 5,
   and a second binding region binding to human CD20 which comprises the CDR sequences:
   VH-CDR1: SEQ ID NO: 8
   VH-CDR2: SEQ ID NO:9
   VH-CDR3: SEQ ID NO: 10
   VL-CDR1: SEQ ID NO: 11
   VL-CDR2: DAS, and
   VL-CDR3: SEQ ID NO: 12
   in an amount of from about 5 μg to about 50 mg,
b. acetate buffer and sorbitol in a ratio of between 1:5 and 1:10 wherein the osmolality of the unit dosage form is from about 210 to about 250 and the pH is about 5.5.

In yet a another aspect the invention relates to a unit dosage form, comprising
a. a bispecific antibody comprising a first binding region binding to human CD3 which comprises the CDR sequences:
   VH-CDR1: SEQ ID NO: 1
   VH-CDR2: SEQ ID NO: 2
   VH-CDR3: SEQ ID NO:3
   VL-CDR1: SEQ ID NO: 4
   VL-CDR2: GTN, and
   VL-CDR3: SEQ ID NO: 5,
   and a second binding region binding to human CD20 which comprises the CDR sequences:
   VH-CDR1: SEQ ID NO: 8
   VH-CDR2: SEQ ID NO: 9
   VH-CDR3: SEQ ID NO: 10
   VL-CDR1: SEQ ID NO: 11
   VL-CDR2: DAS, and
   VL-CDR3: SEQ ID NO: 12
   in an amount of from about 5 μg to about 50 mg,
b. acetate buffer at a concentration of about 30 mM,
c. sorbitol at a concentration of about 150 mM,
   at a pH of about 5.5.

These and other aspects and embodiments are described in more detail in the following sections.

### BRIEF DESCRIPTION OF THE DRAWINGS

FIG. 1. Solubility screening of Duobody-CD3xCD20 in different formulations. Duobody-CD3xCD20 was formulated in the indicated buffers and consecutively concentrated using centrifugal concentrators with timed spin intervals. The concentration of each formulation was measured after the spin intervals of 20, 50, 60 and 90 min.

FIG. 2. Viscosity of Duobody-CD3xCD20 (120-150 mg/mL) in indicated formulations. The viscosity (cP) of the concentrated Duobody-CD3xCD20 samples (120-150 mg/mL) was measured at varying shear rates in the indicated formulations using a Wells-Brookfield Cone/Plate Rheometer.

FIG. 3. Mean cytokine levels per group in blood from cynomolgus monkeys which received either a single IV dose (0.1 or 1 mg/kg) or a single SC dose (0.1 or 1 mg/kg) of DuoBody-CD3xCD20.

FIG. 4. Effect of 4× repeat IV dosing of DuoBody-CD3xCD20 on B cells in the peripheral blood of cynomolgus monkeys. (A) Mean B cell count (CD4-CD8-CD16-CD19+ cells) over time in the peripheral blood of cynomolgus monkeys after four weekly IV doses (0.01, 0.1 or 1 mg/kg) of DuoBody-CD3xCD20, per dose group. (B) Mean B cell counts per dose group as percentage of the B cell counts prior to dosing. B cell counts are shown as absolute cell numbers (cells/μL). 5. Effect of a single SC dose of DuoBody-CD3xCD20 on B cells in the peripheral blood of cynomolgus monkeys. (A) Mean B cell count over time in the peripheral blood of cynomolgus monkeys after a single SC dose (0.01, 0.1, 1, 10 or 20 mg/kg) of DuoBody-CD3xCD20, per dose group. (B) Mean B cell counts per dose group as percentage of the B cell counts prior to dosing. B cell counts are shown as absolute cell numbers (cells/μL).

FIG. 6: Effect of IV infusion of a priming dose followed by target dose of DuoBody-CD3xCD20 on B cells in the peripheral blood of cynomolgus monkeys. Mean B cell counts (CD4-CD8-CD16-CD19+ cells) over time in peripheral blood of cynomolgus monkeys dosed with an IV infusion as priming dose (0.01 mg/kg) followed one day later by one target dose (1 mg/kg; IV). B cell counts are shown as absolute cell numbers (cells/μL).

7. Effect of 4× repeat IV dosing of DuoBody-CD3xCD20 on B cells in the lymph nodes of cynomolgus monkeys. (A) Mean B frequency (CD4-CD8-CD16-CD19+ cells as a percentage of the total lymphocyte population) over time in lymph nodes of cynomolgus monkeys after four weekly IV doses (0.01, 0.1 or 1 mg/kg) of DuoBody-CD3xCD20, per dose group. (B) Mean B cell frequency per dose group as percentage of the B cell frequency prior to dosing.

8: Effect of a single SC dose of DuoBody-CD3xCD20 on B cells in the lymph nodes of cynomolgus monkeys. (A) Mean B cell frequency (CD4-CD8-CD16-CD19+ cells as a percentage of the total lymphocyte population) over time in lymph nodes of cynomolgus monkeys after a single SC dose (0.01, 0.1, 1, 10 or 20 mg/kg) of DuoBody-CD3xCD20, per dose group. (B) Mean B cell frequency per dose group as percentage of the B cell frequency prior to dosing.

9: Effect of IV infusion of a priming dose followed by target dose of DuoBody-CD3xCD20 on B cells in the lymph nodes of cynomolgus monkeys. Mean B cell frequency (CD4-CD8-CD16-CD19+ cells as a percentage of the total lymphocyte population) over time in lymph nodes of cynomolgus monkeys dosed with an IV infusion as priming dose (0.01 mg/kg) followed one day later by one target dose (1 mg/kg; IV).

10: B cell depletion and recovery in spleen and lymph nodes of cynomolgus monkeys following IV treatment with DuoBody-CD3xCD20. Upper panels: cynomolgus monkey 1 was treated with 0.01 mg/kg DuoBody-CD3xCD20 as a priming dose at day 1 and a 1 mg/kg target dose at day 2. The animal was euthanized on day 29 according to schedule. Peripheral blood B cell counts had not recovered at the time of necropsy. Lower panels: cynomolgus monkey 2 was treated with 4 weekly 1 mg/kg doses of DuoBody-Cd3xCD20. The animal was euthanized on day 148 after B cell recovery was observed in peripheral blood. Frozen sections of lymph node and spleen were stained using a CD19-specific antibody to detect B cells (brown staining). Haematoxylin was used to detect cell nuclei (blue staining).

11: Effect of 5× repeat IV dosing of DuoBody-CD3xCD20 on B cells in the peripheral blood of male cynomolgus monkeys. (A) Mean B cell numbers (CD45+CD4-CD8-CD16-CD19+ cells) over time in the peripheral blood of male cynomolgus monkeys after five weekly IV doses of saline or 0.01, 0.1 or 1 mg/kg of DuoBody-CD3xCD20, per dose group. (B) Mean B cell numbers per dose group as percentage of the B cell counts prior to dosing. B cell counts are shown as % of gated lymphocytes.

12: Effect of 5× repeat IV dosing of DuoBody-CD3xCD20 on B cells in the peripheral blood of female cynomolgus monkeys. (A) Mean B cell numbers (CD45+CD4-CD8-CD16-CD19+ cells) over time in peripheral blood of female cynomolgus monkeys after five weekly IV doses of saline or 0.01, 0.1 or 1 mg/kg of DuoBody-CD3xCD20, per dose group. (B) Mean B cell numbers per dose group as percentage of the B cell counts prior to dosing. B cell counts are shown as % of gated lymphocytes.

13: Effect of single IV infusion of DuoBody-CD3xCD20 on B cells in the peripheral blood of male cynomolgus monkeys. (A) Mean B cell numbers (CD45+CD4-CD8-CD16-CD19+ cells) overtime in peripheral blood of male cynomolgus monkeys after a single IV infusion of 0.1 or 1 mg/kg of DuoBody-CD3xCD20, per dose group. (B) Mean B cell numbers per dose group as percentage of the B cell counts prior to dosing. B cell counts are shown as % of gated lymphocytes.

14: Effect of single IV infusion of DuoBody-CD3xCD20 on B cells in the peripheral blood of female cynomolgus monkeys. (A) Mean B cell numbers (CD45+CD4-CD8-CD16-CD19+ cells) overtime in peripheral blood of female cynomolgus monkeys after a single IV infusion of 0.1 or 1 mg/kg of DuoBody-CD3xCD20, per dose group. (B) Mean B cell numbers per dose group as percentage of the B cell counts prior to dosing. B cell counts are shown as % of gated lymphocytes.

15. Effect of SC injection of DuoBody-CD3xCD20 on B cells in the peripheral blood of male cynomolgus monkeys. (A) Mean B cell numbers (CD45+CD4-CD8-CD16-CD19+ cells) overtime in peripheral blood of male cynomolgus monkeys after SC injection of 0.1, 1 or 10 mg/kg of DuoBody-CD3xCD20, per dose group. (B) Mean B cell numbers per dose group as percentage of the B cell counts prior to dosing. B cell counts are shown as % of gated lymphocytes.

1. Effect of SC injection of DuoBody-CD3xCD20 on B cells in the peripheral blood of female cynomolgus monkeys. (A) Mean B cell numbers (CD45+CD4-CD8-CD16-CD19+ cells) overtime in peripheral blood of female cynomolgus monkeys after SC injection of 0.1, 1 or 10 mg/kg of DuoBody-CD3xCD20, per dose group. (B) Mean B cell numbers per dose group as percentage of the B cell counts prior to dosing. B cell counts are shown as % of gated lymphocytes.

FIG. 17. (A) Individual plasma concentration profiles in cynomolgus monkeys following IV administration of DuoBody-CD3xCD20. (B) Individual plasma concentration profiles in cynomolgus monkeys following SC administration of DuoBody-CD3xCD20. Plasma concentration profiles for DuoBody-CD3xCD20 were measured after SC single dose injection of DuoBody-CD3xCD20 at dose levels of 0.01, 0.1, 1, 10, or 20 mg/kg. (C) Group mean plasma concentration profiles for cynomolgus monkeys after either IV infusion or SC injection.

### DETAILED DESCRIPTION OF THE INVENTION

### Definitions

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH or V_{H}) and a heavy chain constant region (abbreviated herein as CH or C_{H}). The heavy chain constant region typically is comprised of three domains, CH1, CH2, and CH3. The hinge region is the region between the CH1 and CH2 domains of the heavy chain and is highly flexible. Disulphide bonds in the hinge region are part of the interactions between two heavy chains in an IgG molecule. Each light chain typically is comprised of a light chain variable region (abbreviated herein as VL or V_{L}) and a light chain constant region (abbreviated herein as CL or C_{L}). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Unless otherwise stated or contradicted by context, CDR sequences herein are identified according to IMGT rules (Brochet X., Nucl Acids Res. 2008; 36: W503-508 and Lefranc M P., Nucleic Acids Research 1999; 27:209-212; see also internet http address http://www.imgt.org/). Unless otherwise stated or contradicted by context, reference to amino acid positions in the constant regions in the present invention is according to the EU-numbering (Edelman et al., Proc Natl Acad Sci USA. 1969 May; 63(1):78-85; Kabat et al., Sequences of Proteins of Immunological Interest, Fifth Edition. 1991 NIH Publication No. 91-3242). For example, SEQ ID NO: 15 herein sets forth amino acids positions 118-447 according to EU numbering, of the IgG1 heavy chain constant region.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is considered well-known in the art how to align a sequence or segment in a sequence and thereby determine the corresponding position in a sequence to an amino acid position according to the present invention.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term “antibody-binding region”, as used herein, refers to the region which interacts with the antigen and comprises both the VH and the VL regions. The term antibody when used herein comprises not only monospecific antibodies, but also multispecific antibodies which comprise multiple, such as two or more, e.g. three or more, different antigen-binding regions. The constant regions of the antibodies (Abs) may mediate the binding of the immunoglobulin to host tissues or factors, including various cells of the immune system (such as effector cells) and components of the complement system such as C1q, the first component in the classical pathway of complement activation. As indicated above, the term antibody herein, unless otherwise stated or clearly contradicted by context, includes fragments of an antibody that are antigen-binding fragments, i.e., retain the ability to specifically bind to the antigen. It has been shown that the antigen-binding function of an antibody may be performed by fragments of a full-length antibody. Examples of antigen-binding fragments encompassed within the term “antibody” include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al; Trends Biotechnol. 2003 November; 21(11):484-90); (vi) camelid or nanobodies (Revets et al; Expert Opin Biol Ther. 2005 January; 5(1):111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention, as well as bispecific formats of such fragments, are discussed further herein. It also should be understood that the term antibody, unless specified otherwise, also includes polyclonal antibodies, monoclonal antibodies (mAbs), antibody-like polypeptides, chimeric antibodies and humanized antibodies, and antibody fragments retaining the ability to specifically bind to the antigen (antigen-binding fragments) provided by any known technique, such as enzymatic cleavage, peptide synthesis, and recombinant techniques. An antibody as generated can possess any isotype. As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g. IgG1, is mentioned herein, the term is not limited to a specific isotype sequence, e.g. a particular IgG1 sequence, but is used to indicate that the antibody is closer in sequence to that isotype, e.g. IgG1, than to other isotypes. Thus, e.g. an IgG1 antibody of the invention may be a sequence variant of a naturally-occurring IgG1 antibody, including variations in the constant regions.

The term “monoclonal antibody” as used herein refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

The term “bispecific antibody” or “bs” or “bsAb” in the context of the present invention refers to an antibody having two different antigen-binding regions defined by different antibody sequences. A bispecific antibody can be of any format.

When used herein, the terms “half molecule”, “Fab-arm” and “arm” refer to one heavy chain-light chain pair.

When a bispecific antibody is described to comprise a half-molecule antibody “derived from” a first antibody, and a half-molecule antibody “derived from” a second antibody, the term “derived from” indicates that the bispecific antibody was generated by recombining, by any known method, said half-molecules from each of said first and second antibodies into the resulting bispecific antibody. In this context, “recombining” is not intended to be limited by any particular method of recombining and thus includes all of the methods for producing bispecific antibodies described herein below, including for example recombining by half-molecule exchange (also known as “controlled Fab-arm exchange”), as well as recombining at nucleic acid level and/or through co-expression of two half-molecules in the same cells.

The term “monovalent antibody” means in the context of the present invention that an antibody molecule is capable of binding a single molecule of an antigen, and thus is not capable of crosslinking antigens or cells.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

When used herein, unless contradicted by context, the term “Fc region” refers to an antibody region consisting of the Fc sequences of the two heavy chains of an immunoglobulin, wherein said Fc sequences comprise at least a hinge region, a CH2 domain, and a CH3 domain.

When used herein the term “heterodimeric interaction between the first and second CH3 regions” refers to the interaction between the first CH3 region and the second CH3 region in a first-CH3/second-CH3 heterodimeric protein.

When used herein the term “homodimeric interactions of the first and second CH3 regions” refers to the interaction between a first CH3 region and another first CH3 region in a first-CH3/first-CH3 homodimeric protein and the interaction between a second CH3 region and another second CH3 region in a second-CH3/second-CH3 homodimeric protein.

As used herein, the term “binding” in the context of the binding of an antibody to a predetermined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific). The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

In a preferred embodiment, the antibody of the invention is isolated. An “isolated antibody” as used herein, is intended to refer to an antibody which is substantially free of other antibodies having different antigenic specificities. In a preferred embodiment, an isolated bispecific antibody that specifically binds to CD20 and CD3 is in addition substantially free of monospecific antibodies that specifically bind to CD20 or CD3.

The term “CD3” as used herein, refers to the human Cluster of Differentiation 3 protein which is part of the T-cell co-receptor protein complex and is composed of four distinct chains. CD3 is also found in other species, and thus, the term “CD3” is not limited to human CD3 unless contradicted by context. In mammals, the complex contains a CD3γ (gamma) chain (human CD3γ chain UniProtKB/Swiss-Prot No P09693, or cynomolgus monkey CD3γ UniProtKB/Swiss-Prot No Q95LI7), a CD3δ (delta) chain (human CD3δ UniProtKB/Swiss-Prot No P04234, or cynomolgus monkey CD3δ UniProtKB/Swiss-Prot No Q95LI8), two CD3ε (epsilon) chains (human CD3ε UniProtKB/Swiss-Prot No P07766; cynomolgus CD3ε UniProtKB/Swiss-Prot No Q95LI5; or rhesus CD3ε UniProtKB/Swiss-Prot No G7NCB9), and a CD3ζ-chain (zeta) chain (human CD3ζ UniProtKB/Swiss-Prot No P20963, cynomolgus monkey CD3ζ UniProtKB/Swiss-Prot No Q09TK0). These chains associate with a molecule known as the T-cell receptor (TCR) and generate an activation signal in T lymphocytes. The TCR and CD3 molecules together comprise the TCR complex.

A “CD3 antibody” or “anti-CD3 antibody” is an antibody which binds specifically to the antigen CD3, in particular human CD3ε (epsilon).

The term “human CD20” or “CD20” refers to human CD20 (UniProtKB/Swiss-Prot No P11836) and includes any variants, isoforms and species homologs of CD20 which are naturally expressed by cells, including tumor cells, or are expressed on cells transfected with the CD20 gene or cDNA. Species homologs include rhesus monkey CD20 (Macaca mulatta; UniProtKB/Swiss-Prot No H9YXP1) and cynomolgus monkey CD20 (Macaca fascicularis; UniProtKB No G7PQ03).

A “CD20 antibody” or “anti-CD20 antibody” is an antibody which binds specifically to the antigen CD20, in particular to human CD20.

A “CD3xCD20 antibody”, “anti-CD3xCD20 antibody”, “CD20xCD3 antibody” or “anti-CD20xCD3 antibody” is a bispecific antibody, which comprises two different antigen-binding regions, one of which binds specifically to the antigen CD20 and one of which binds specifically to CD3. As used herein the term “DuoBody-CD3xCD20” refers to an IgG1 bispecific CD3xCD20 antibody wherein the CD3 binding Fab-arm comprise the VH and VL sequences as defined in SEQ ID Nos 6 and 7, respectively, the constant light chain sequence as defined in SEQ ID NO: 22, and the constant heavy chain sequence as defined in SEQ ID NO: 19 (FEAL) and wherein the CD20 binding Fab-arm comprise the VH and VL sequences of SEQ ID: 13 and 14, respectively, the constant light chain sequence as defined in SEQ ID NO: 23, and the constant heavy chain sequence as defined in SEQ ID NO: 20 (FEAR)”. This bispecific antibody may be prepared as described in WO 2016/110576.

In a preferred embodiment, the bispecific antibody of the invention is isolated. An “isolated bispecific antibody,” as used herein, is intended to refer to a bispecific antibody which is substantially free of other antibodies having different antigenic specificities (for instance an isolated bispecific antibody that specifically binds to CD20 and CD3 is substantially free of monospecific antibodies that specifically bind to CD20 or CD3).

The present invention also provides antibodies comprising functional variants of the VL regions, VH regions, or one or more CDRs of the antibodies of the examples. A functional variant of a VL, VH, or CDR used in the context of an antibody still allows the antibody to retain at least a substantial proportion (at least about 50%, 60%, 70%, 80%, 90%, 95% or more) of the affinity and/or the specificity/selectivity of the “reference” or “parent” antibody and in some cases, such an antibody may be associated with greater affinity, selectivity and/or specificity than the parent antibody.

Such functional variants typically retain significant sequence identity to the parent antibody. The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

Exemplary variants include those which differ from VH and/or VL and/or CDR regions of the parent antibody sequences mainly by conservative substitutions; for instance, 10, such as 9, 8, 7, 6, 5, 4, 3, 2 or 1 of the substitutions in the variant are conservative amino acid residue replacements.

In the context of the present invention, conservative substitutions may be defined by substitutions within the classes of amino acids reflected in the following table:

Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

In the context of the present invention the following notations are, unless otherwise indicated, used to describe a mutation; i) substitution of an amino acid in a given position is written as e.g. K409R which means a substitution of a Lysine in position 409 with an Arginine; and ii) for specific variants the specific three or one letter codes are used, including the codes Xaa and X to indicate any amino acid residue. Thus, the substitution of Lysine with Arginine in position 409 is designated as: K409R, and the substitution of Lysine with any amino acid residue in position 409 is designated as K409X. In case of deletion of Lysine in position 409 it is indicated by K409*.

In the context of the present invention, “competition” (or “blocking” or “cross-blocking”) refers to a significant reduction in the propensity for a particular molecule to bind a particular binding partner in the presence of another molecule that binds the binding partner. Competition for binding to CD20 by two or more anti-CD20 antibodies may be determined by any suitable technique.

The term “epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding and other amino acid residues, which are not directly involved in the binding, such as amino acid residues which are effectively blocked or covered by the specifically antigen binding peptide (in other words, the amino acid residue is within the footprint of the specifically antigen binding peptide).

The term “chimeric antibody” as used herein, refers to an antibody wherein the variable region is derived from a non-human species (e.g. derived from rodents) and the constant region is derived from a different species, such as human. Chimeric monoclonal antibodies for therapeutic applications are developed to reduce antibody immunogenicity. The terms “variable region” or “variable domain” as used in the context of chimeric antibodies, refer to a region which comprises the CDRs and framework regions of both the heavy and light chains of the immunoglobulin. Chimeric antibodies may be generated by using standard DNA techniques as described in Sambrook et al., 1989, Molecular Cloning: A laboratory Manual, New York: Cold Spring Harbor Laboratory Press, Ch. 15. The chimeric antibody may be a genetically or an enzymatically engineered recombinant antibody. It is within the knowledge of the skilled person to generate a chimeric antibody, and thus, generation of the chimeric antibody according to the present invention may be performed by other methods than described herein.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “human antibody” as used herein, refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. Human monoclonal antibodies of the invention can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known. Human monoclonal antibodies can thus e.g. be generated using transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery, see e.g. Mendez et al. 1997 Nat Genet. 15(2):146-56. The term “reducing conditions” or “reducing environment” refers to a condition or an environment in which a substrate, here a cysteine residue in the hinge region of an antibody, is more likely to become reduced than oxidized.

The term “recombinant host cell” (or simply “host cell”), as used herein, is intended to refer to a cell into which an expression vector has been introduced, e.g. an expression vector encoding an antibody of the invention. Recombinant host cells include, for example, transfectomas, such as CHO, CHO-S, HEK, HEK293, HEK-293F, Expi293F, PER.C6 or NSO cells, and lymphocytic cells.

The term “treatment” refers to the administration of an effective amount of a therapeutically active antibody of the present invention with the purpose of easing, ameliorating, arresting or eradicating (curing) symptoms or disease states.

The term “effective amount” or “therapeutically effective amount” refers to an amount effective, at dosages and for periods of time necessary, to achieve a desired therapeutic result.

A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

The term “buffer” as used herein denotes a pharmaceutically acceptable buffer. The term “buffer” encompasses those agents which maintain the pH value of a solution, e.g., in an acceptable range and includes, but is not limited to, acetate, histidine, TRIS® (tris (hydroxymethyl) aminomethane), citrate, succinate, glycolate and the like. Generally, the “buffer” as used herein has a pKa and buffering capacity suitable for the pH range of about 5 to about 6, preferably of about 5.5.

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/w or less, such as 3% w/w or less, such as 1% w/w or less). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleate, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl hydroxystearate, polyoxylglycerides, polysorbates, propylene glycol dilaurate, propylene glycol monolaurate, sorbitan esters sucrose palmitate, sucrose stearate, tricaprylin and TPGS (Nonionic and zwitterionic surfactants).

A “diluent” of interest herein is one which is pharmaceutically acceptable (safe and non-toxic for administration to a human) and is useful for the preparation of dilutions of the pharmaceutical composition. Preferably such dilutions of the composition of the invention dilute only the antibody concentration but not the buffer and stabilizer. Accordingly, in a preferred embodiment the diluent contains the same concentrations of the buffer and stabilizer as is present in the pharmaceutical composition of the invention. Further exemplary diluents include sterile water, bacteriostatic water for injection (BWFI), a pH buffered solution which is preferably an acetate buffer, sterile saline solution, Ringer's solution or dextrose solution. In a preferred embodiment the diluent comprise or consist essentially of acetate buffer and sorbitol.

The terms “pharmaceutical composition” and “pharmaceutical formulation” is used interchangeably herein.

### Specific Embodiments of the Invention

In a main aspect the present invention provides a pharmaceutical composition comprising:
a. about 50 to about 120 mg/mL of a bispecific antibody binding to human CD3 and human CD20,
b. about 20 to about 40 mM acetate,
c. about 140 to about 160 mM sorbitol

where the pH of the composition is about 5 to about 6 and where the bispecific antibody comprises a first binding region binding to human CD3 which comprises the CDR sequences: VH-CDR1: SEQ ID NO: 1, VH-CDR2: SEQ ID NO: 2, VH-CDR3: SEQ ID NO: 3, VL-CDR1: SEQ ID NO: 4, VL-CDR2: GTN, and VL-CDR3: SEQ ID NO: 5, and a second binding region binding to human CD20 which comprises the CDR sequences: VH-CDR1: SEQ ID NO: 8, VH-CDR2: SEQ ID NO: 9, VH-CDR3: SEQ ID NO: 10, VL-CDR1: SEQ ID NO: 11, VL-CDR2: DAS, and VL-CDR3: SEQ ID NO: 12.

In another aspect the present invention provides a pharmaceutical composition consisting essentially of:
a. about 50 to about 120 mg/mL of a bispecific antibody binding to human CD3 and human CD20,
b. about 20 to about 40 mM acetate,
c. about 140 to about 160 mM sorbitol

where the pH of the composition is about 5 to about 6 and where the bispecific antibody comprises a first binding region binding to human CD3 which comprises the CDR sequences: VH-CDR1: SEQ ID NO: 1, VH-CDR2: SEQ ID NO: 2, VH-CDR3: SEQ ID NO: 3, VL-CDR1: SEQ ID NO: 4, VL-CDR2: GTN, and VL-CDR3: SEQ ID NO: 5, and a second binding region binding to human CD20 which comprises the CDR sequences: VH-CDR1: SEQ ID NO: 8, VH-CDR2: SEQ ID NO: 9, VH-CDR3: SEQ ID NO: 10, VL-CDR1: SEQ ID NO: 11, VL-CDR2: DAS, and VL-CDR3: SEQ ID NO: 12. Hereby a simple yet stable pharmaceutical composition is provided. It is an advantage of the present invention that the composition is suitable both for IV administration and for SC administration. It is further advantage that the composition is stable and in particular that the bispecific antibody is stable over a broad range of antibody concentrations so that the same formulation may be used for clinical trial phase I dose escalation studies where the antibody concentration in the composition varies from about as low as 4 μg/mL to as high as 120 mg/mL or even higher and the same composition may be used for later stages of clinical trials and even for the final commercial formulation. It is surprising that such a formulation is stable over such a broad range of antibody concentrations at temperatures varying from 2° to 25° C. or even higher temperatures. The composition of the invention is stable for at least 3 months, such as at least 6 months, or even for at least 9 months or for at least 12 months when stored at between 2° C. and 8° C.

In an embodiment of the composition of the invention the first binding region of the bispecific antibody binding to CD3 comprises the VH and VL sequences of SEQ ID NOs: 6 and 7.

In a further embodiment of the composition of the invention the second binding region of the bispecific antibody binding to CD20 comprises the VH and VL sequences of SEQ ID: 13 and 14.

In a further embodiment of the pharmaceutical composition of the invention the bispecific antibody is DuoBody-CD3xCD20.

In a preferred embodiment of the composition of the invention the bispecific antibody is an IgG1 antibody. However, the bispecific antibody may alternatively be an IgG2, IgG3 or IgG4 antibody isotype or a combination of IgG1, IgG2, IgG3 or IgG4. For instance for first heavy chain could be IgG1 isotype and the second heavy chain could be IgG4 isotype.

In a further embodiment of the composition of the invention the bispecific antibody comprises an Fc region which comprises a first and second heavy chain, wherein said Fc region has been modified so that it has reduced effector functions compared to the bispecific antibody comprising a wild-type IgG1 Fc region. Hereby the bispecific antibody will have reduced ability to bind to human Fcgamma receptors and human complement component C1q, resulting in reduced ability to induce Fc-mediated effector functions such as antibody-dependent cell-mediated cytotoxicity (ADCC), antibody-dependent cell-mediated phagocytosis (ADCP) and complement-dependent cytotoxicity (CDC). Accordingly, a bispecific antibody of the invention which has reduced effector functions only activates T cells in the presence of CD20 expressing cells. In other words, such bispecific antibodies will not induce antibody-mediated, FcR-dependent CD3 crosslinking and subsequent target-independent T-cell activation.

In another embodiment of the pharmaceutical composition of the invention the bispecific antibody comprises an Fc region which has been modified so that binding of C1q to said antibody is reduced compared to the bispecific antibody having a wild-type IgG1 Fc region by at least 70%, at least 80%, at least 90%, at least 95%, at least 97%, or 100%, wherein C1q binding is determined by ELISA.

A bispecific antibody as described herein can be generated according to the DuoBody® technology platform (Genmab A/S) as described, e.g., in WO 2011/131746 and in Labrijn A F et al., (2013) PNAS 110(13): 5145-5150. The DuoBody technology can be used to combine one half of a first monospecific antibody containing two heavy and two light chains with one half of a second monospecific antibody containing two heavy and two light chains. The resultant heterodimer contains one heavy chain and one light chain from the first antibody paired with one heavy chain and one light chain from the second antibody. When the first and the second monospecific antibodies recognize different epitopes on different antigens, such as CD3 and CD20, the resultant heterodimer is a bispecific antibody against CD3 and CD20.

The DuoBody technology requires that each of the monospecific antibodies includes a heavy chain constant region with a single point mutation in the CH3 domain. The point mutations allow for a stronger interaction between the CH3 domains in the resultant bispecific antibody than between the CH3 domains in either of the monospecific antibodies. The single point mutation in each monospecific antibody is at residue 366, 368, 370, 399, 405, 407, or 409 in the CH3 domain of the heavy chain constant region using the EU-index for numbering, as described, e.g., in WO 2011/131746. Moreover, the single point mutation is located at a different residue in one monospecific antibody as compared to the other monospecific antibody. For example, one monospecific antibody can comprise the mutation F405L (i.e., a mutation from phenylalanine to leucine at residue 405), while the other monospecific antibody can comprise the mutation K409R (i.e., a mutation from lysine to arginine at residue 409). The heavy chain constant regions of the monospecific antibodies can be an IgG1, IgG2, IgG3, or IgG4 isotype (e.g., a human IgG1 isotype), and a bispecific antibody produced by the DuoBody technology can retain Fc-mediated effector functions or the Fc region may be further mutated to reduce the Fc-mediated effector functions as described herein.

Accordingly, in another embodiment of the pharmaceutical composition of the invention the bispecific antibody comprises a first and second heavy chain each comprising at least a hinge region, a CH2 and CH3 region, wherein in said first heavy chain at least one of the amino acids in the positions corresponding to a positions selected from the group consisting of T366, L368, K370, D399, F405, Y407, and K409 in a human IgG1 heavy chain has been substituted, and in said second heavy chain at least one of the amino acids in the positions corresponding to a position selected from the group consisting of T366, L368, K370, D399, F405, Y407, and K409 (according to the EU numbering system) in a human IgG1 heavy chain has been substituted, and wherein said first and said second heavy chains are not substituted in the same positions.

In yet another embodiment of the pharmaceutical composition of the invention (i) the amino acid in the position corresponding to F405 in a human IgG1 heavy chain is substituted with L in said first heavy chain of the bispecific antibody, and the amino acid in the position corresponding to K409 in a human IgG1 heavy chain is substituted with R in said second heavy chain of the bispecific antibody, or (ii) the amino acid in the position corresponding to K409 in a human IgG1 heavy chain is R in said first heavy chain, and the amino acid in the position corresponding to F405 in a human IgG1 heavy chain is L in said second heavy chain.

In an embodiment the bispecific antibody of the pharmaceutical composition may further be substituted in both the first constant heavy chain and the second constant heavy chain of the bispecific antibody in the positions corresponding to positions L234 and L235 in the human IgG1 heavy chain (EU index numbering) so that L234 is substituted with an F (L234F) and L235 is substituted with an E (L235E). Hereby the Fc-mediated effector functions of the antibody are reduced.

In a further embodiment the bispecific antibody of the pharmaceutical composition may further be substituted in both the first constant heavy chain and the second constant heavy chain of the bispecific antibody in the position corresponding to D265 in the human IgG1 so that D265 is substituted with an A (D265A).

In another embodiment the bispecific antibody of the pharmaceutical composition comprises the three substitutions L234F+L235E+D265A in both the first and the second constant heavy chains of the bispecific antibody.

In another embodiment the bispecific antibody of the pharmaceutical composition comprises the three substitutions L234F+L235E+D265A in both the first and the second constant heavy chains of the bispecific antibody and the first constant heavy chain further comprise an F405L substitution, and the second constant heavy chain further comprise a K409R substitution or vice versa. Hereby the first constant heavy chain comprise the substitutions L234F+L235E+D265A+F405L (also described as “FEAL” mutations herein) and the second constant heavy chain comprise the substitutions L234F+L235E+D265A+K409R (also described as “FEAR” mutations herein) or the first constant heavy chain comprise the substitutions L234F+L235E+D265A+K409R and the second constant heavy chain comprise the substitutions L234F+L235E+D265A+F405L. In a preferred embodiment the first and the second constant heavy chains of the bispecific antibody are of IgG1 isotype but comprising the substitutions L234F+L235E+D265A+F405L and L234F+L235E+D265A+K409R, respectively.

Accordingly, in an embodiment of the invention, the bispecific antibody of the pharmaceutical composition comprises a first heavy chain constant region of SEQ ID NO: 19 and a second heavy chain constant region of SEQ ID NO: 20 or it comprises a first heavy chain constant region of SEQ ID NO: 20 and a second heavy chain constant region of SEQ ID NO: 19. In another embodiment the bispecific antibody comprises heavy chain constant regions that have at least 90% sequence identity, such as at least 91%, such as at least 92%, such as at least 93%, such as at least 94%, such as at least 95%, such as at least 96%, such as at least 97%, such as at least 98%, such as at least 99% sequence identity to the amino acid sequences of SEQ ID NO 19 and 20 respectively, but comprising the FEAR and FEAL amino acids as described above.

The first and second light chains of the bispecific antibody of the composition preferably further comprise a first and second light chain constant region. The light chain constant region may be of lambda or kappa subtype. In a preferred embodiment of the invention the constant region of the light chain of the CD3 binding arm is of lambda subtype and the constant region of the light chain of the CD20 binding arm is of kappa subtype. In one embodiment the light chain of the CD3 binding arm has the sequence of SEQ ID NO: 24 and the light chain of the CD20 binding arm has the sequence of SEQ ID NO:25.

The concentration of the bispecific antibody of the pharmaceutical composition may be from about 1 mg/mL to about 200 mg/mL. In an embodiment of the invention the concentration of the bispecific antibody is from about 50 to about 120 mg/mL. In another embodiment of the invention the concentration of the bispecific antibody is from about 50 to about 110 mg/mL. In another embodiment of the invention the concentration of the bispecific antibody is from about 50 to about 100 mg/mL. In another embodiment of the invention the concentration of the bispecific antibody is from about 50 to about 90 mg/mL. In another embodiment of the invention the concentration of the bispecific antibody is from about 50 to about 80 mg/mL. In another embodiment of the invention the concentration of the bispecific antibody is from about 50 to about 70 mg/mL. In another embodiment of the invention the concentration of the bispecific antibody is about 60 mg/mL. In another embodiment of the invention the concentration of the bispecific antibody is about 70 mg/mL. In another embodiment of the invention the concentration of the bispecific antibody is about 80 mg/mL. In another embodiment of the invention the concentration of the bispecific antibody is about 90 mg/mL. In another embodiment of the invention the concentration of the bispecific antibody is about 100 mg/mL. In another embodiment of the invention the concentration of the bispecific antibody is about 110 mg/mL. In another embodiment of the invention the concentration of the bispecific antibody is about 120 mg/mL. In another embodiment of the invention the concentration of the bispecific antibody is about 130 mg/mL. In another embodiment of the invention the concentration of the bispecific antibody is about 140 mg/mL. In another embodiment of the invention the concentration of the bispecific antibody is about 150 mg/mL.

The concentration of the bispecific antibody of the pharmaceutical composition may be from 1 mg/mL to 200 mg/mL. In an embodiment of the invention the concentration of the bispecific antibody in the pharmaceutic composition is from 50 to 120 mg/mL. In another embodiment of the invention the concentration of the bispecific antibody is from 50 to 110 mg/mL. In another embodiment of the invention the concentration of the bispecific antibody is from 50 to 100 mg/mL. In another embodiment of the invention the concentration of the bispecific antibody is from 50 to 90 mg/mL. In another embodiment of the invention the concentration of the bispecific antibody is from 50 to 80 mg/mL. In another embodiment of the invention the concentration of the bispecific antibody is from 50 to 70 mg/mL. In another embodiment of the invention the concentration of the bispecific antibody in the pharmaceutical composition is 60 mg/mL. In another embodiment of the invention the concentration of the bispecific antibody in the pharmaceutical composition is 70 mg/mL. In another embodiment of the invention the concentration of the bispecific antibody in the pharmaceutical composition is 80 mg/mL. In another embodiment of the invention the concentration of the bispecific antibody in the pharmaceutical composition is 90 mg/mL. In another embodiment of the invention the concentration of the bispecific antibody in the pharmaceutical composition is 100 mg/mL. In another embodiment of the invention the concentration of the bispecific antibody in the pharmaceutical composition is 110 mg/mL. In another embodiment of the invention the concentration of the bispecific antibody in the pharmaceutical composition is 120 mg/mL. In another embodiment of the invention the concentration of the bispecific antibody in the pharmaceutical composition is 130 mg/mL. In another embodiment of the invention the concentration of the bispecific antibody in the pharmaceutical composition is 140 mg/mL. In another embodiment of the invention the concentration of the bispecific antibody in the pharmaceutical composition is 150 mg/mL.

The pharmaceutical composition of the invention comprises an acetate buffer which is used to control the pH in a range which optimizes the therapeutic effectiveness and the stability of the bispecific antibody. The acetate buffer may be produced mixing sodium acetate trihydrate with acetic acid in water for injection. The pH may be adjusted by adding sodium hydroxide. In one embodiment the acetate buffer is present at concentrations between 20 mM and 40 mM. In one embodiment of the invention the concentration of the acetate buffer in the composition is 20 mM. In another embodiment of the invention the concentration of the acetate buffer in the composition is 25 mM. In another embodiment of the invention the concentration of the acetate buffer in the composition is 30 mM. In another embodiment of the invention the concentration of the acetate buffer in the composition is 35 mM. In another embodiment of the invention the concentration of the acetate buffer in the composition is 40 mM. In one embodiment of the invention the pharmaceutical composition may comprise further buffers. In another embodiment the pharmaceutical composition does not comprise further buffers.

In an embodiment of the invention the pH of the pharmaceutical composition is in the range of 5 to 6. In another embodiment of the invention the pH of the pharmaceutical composition is in the range of 5.2 to 5.8. In another embodiment of the invention the pH of the pharmaceutical composition is in the range of 5.4 to 5.6. In another embodiment of the invention the pH of the pharmaceutical composition is about 5.5 such as 5.5.

The pharmaceutical composition of the invention further comprise sorbitol as a “stabilizer” which can interact with the charged groups of the amino acid side chains, thereby lessening the potential for inter and intra-molecular interactions. In one embodiment sorbitol is present in the pharmaceutical composition at concentrations between 100 mM and 250 mM. In another embodiment sorbitol is present at concentrations between 130 mM and 200 mM. In one embodiment sorbitol is present in the pharmaceutical composition at a concentration of 140 mM. In one embodiment sorbitol is present in the pharmaceutical composition at a concentration of 150 mM. In one embodiment sorbitol is present in the pharmaceutical composition at a concentration of 180 mM. In one embodiment sorbitol is present in the pharmaceutical composition at a concentration of 200 mM. In one embodiment sorbitol is present in the pharmaceutical composition at a concentration of 220 mM. In one embodiment sorbitol is present in the pharmaceutical composition at a concentration of 230 mM. In one embodiment sorbitol is present in the pharmaceutical composition at a concentration of 240 mM. In one embodiment sorbitol is present in the pharmaceutical composition at a concentration of 250 mM.

In one embodiment the osmolality (mOsm/kg) of the pharmaceutical composition is 200 mOsm/kg. In another embodiment the osmolality of the pharmaceutical composition is 210 mOsm/kg. In another embodiment the osmolality of the pharmaceutical composition is 220 mOsm/kg. In another embodiment the osmolality of the pharmaceutical composition is 230 mOsm/kg. In another embodiment the osmolality of the pharmaceutical composition is 240 mOsm/kg. In another embodiment the osmolality of the pharmaceutical composition is 250 mOsm/kg.

In one embodiment of the invention the ratio of the concentrations of acetate buffer to sorbitol in the pharmaceutical composition is between 1:5 and 1:10. In one embodiment of the invention the ratio of the concentrations of acetate buffer to sorbitol is 1:5. In another embodiment of the invention the ratio of the concentrations of acetate buffer to sorbitol is 1:6.

In another embodiment of the invention the ratio of the concentrations of acetate buffer to sorbitol is 1:7. In another embodiment of the invention the ratio of the concentrations of acetate buffer to sorbitol is 1:8. In another embodiment of the invention the ratio of the concentrations of acetate buffer to sorbitol is 1:9. In another embodiment of the invention the ratio of the concentrations of acetate buffer to sorbitol is 1:10.

In an embodiment of the invention the pharmaceutical composition has a pH of about 5.5 and consists essentially of:
a. 50 to 120 mg/mL of the bispecific antibody
b. 20 to 40 mM acetate buffer
c. 140 to 160 mM sorbitol.

In a particular embodiment of the invention the pharmaceutical composition has a pH of about 5.5 and consists essentially of:
a. 60 mg/mL of the bispecific antibody
b. 30 mM acetate buffer
c. 150 mM sorbitol
wherein the CD3 binding Fab-arm of the bispecific antibody comprise the VH and VL sequences as defined in SEQ ID Nos 6 and 7, respectively, and the constant heavy chain sequence as defined in SEQ ID NO: 19 (FEAL) and wherein the CD20 binding Fab-arm comprise the VH and VL sequences of SEQ ID: 13 and 14, respectively, and the constant heavy chain sequence as defined in SEQ ID NO: 20 (FEAR).

In another embodiment of the invention the pharmaceutical composition has a pH of about 5.5 and consists essentially of:
a. 60 mg/mL of the bispecific antibody
b. 30 mM acetate buffer
c. 250 mM sorbitol,
wherein the CD3 binding Fab-arm of the bispecific antibody comprise the VH and VL sequences as defined in SEQ ID Nos 6 and 7, respectively, and the constant heavy chain sequence as defined in SEQ ID NO: 19 (FEAL) and wherein the CD20 binding Fab-arm comprise the VH and VL sequences of SEQ ID: 13 and 14, respectively, and the constant heavy chain sequence as defined in SEQ ID NO: 20 (FEAR).

In one embodiment the pharmaceutical composition is a concentrated drug product (the DuoBody CD3xCD20) formulated in 30 mM acetate, 150 mM sorbitol, pH 5.5. The concentrate may be diluted immediately prior to administration with a diluent resulting in concentrations from 2 μg/mL to 5 mg/mL of the bispecific antibody. In one embodiment the diluent formulation is 30 mM acetate, 150 mM sorbitol, pH 5.5.

In an embodiment of the invention the pharmaceutical composition does not comprise a surfactant. In another embodiment the pharmaceutical composition does not comprise a hyaluronidase. In a further embodiment the pharmaceutical composition does neither comprise a surfactant nor a hyaluronidase.

In a preferred embodiment the pharmaceutical composition is a subcutaneous composition or is a pharmaceutical composition for use in subcutaneous administration. The pharmaceutical composition of the invention may however also be administered intravenously. Accordingly, in one embodiment the pharmaceutical composition is an intravenous composition or the pharmaceutical composition is for use in intravenous administration. It is an advantage of the present invention that the pharmaceutical composition is suitable both for subcutaneous and for intravenous administration.

In an embodiment of the invention the pharmaceutical composition is for use in the treatment of cancer. In an embodiment of the invention the pharmaceutical composition is for use in the treatment of a B-cell malignancy.

In another embodiment, the pharmaceutical composition of the invention can be used to induce T cell-mediated immune responses, inflammation and microenvironment re-modelling.

In a particular embodiment, the pharmaceutical composition is for use in vivo to treat, prevent or diagnose a variety of CD20-related diseases. Examples of CD20-related diseases include, among others, B cell lymphoma, e.g., non-Hodgkin's lymphoma (NHL), B cell leukemia and immune diseases, e.g., autoimmune diseases, such as those listed below.

In one embodiment the pharmaceutical composition according to the invention is for use in the treatment of NHL or B cell leukemia.

In one embodiment, the pharmaceutical composition according to the invention is for use in the treatment of CD20 antibody-resistant NHL or B cell leukemia, such as rituximab- or ofatumumab-resistant NHL or B cell leukemia, e.g. rituximab-resistant non-aggressive B-cell lymphoma.

In one embodiment, the pharmaceutical composition according to the invention is for use in the treatment of Acute Lymphoblastic Leukemia (ALL), such as relapsed or refractory ALL.

In one embodiment, the pharmaceutical composition according to the invention is for use in the treatment of CLL, such as relapsed or refractory CLL.

In one embodiment, the pharmaceutical composition according to the invention is for use in the treatment of FL, such as or relapsed or refractory FL.

The invention further provides a method of treating cancer in a subject comprising administering to a subject in need thereof the pharmaceutical composition as described above for a time sufficient to treat the cancer.

The invention further provides a method of treating cancer in a subject comprising administering to a subject in need thereof the pharmaceutical composition as described above subcutaneously to the subject for a time sufficient to treat the cancer.

The invention further provides a method of treating cancer in a subject comprising administering to a subject in need thereof the pharmaceutical composition as described above intravenously to the subject for a time sufficient to treat the cancer. In an embodiment of the invention the cancer to be treated in this method is a B-cell malignancy such as NHL, CLL, ALL, FL or a CD20 antibody-resistant NHL or B cell leukemia, such as rituximab- or ofatumumab-resistant NHL or B cell leukemia, e.g. rituximab-resistant non-aggressive B-cell lymphoma.

In one embodiment, the pharmaceutical composition according to the invention is in a unit dosage form. In one embodiment the unit dose of the invention is a liquid unit dose.

In one embodiment of the invention the unit dosage form, comprises
a. a bispecific antibody comprising a first binding region binding to human CD3 which comprises the CDR sequences:
   VH-CDR1: SEQ ID NO: 1
   VH-CDR2: SEQ ID NO: 2
   VH-CDR3: SEQ ID NO:3
   VL-CDR1: SEQ ID NO: 4
   VL-CDR2: GTN, and
   VL-CDR3: SEQ ID NO: 5,

and a second binding region binding to human CD20 which comprises the CDR sequences:
VH-CDR1: SEQ ID NO: 8
VH-CDR2: SEQ ID NO: 9
VH-CDR3: SEQ ID NO: 10
VL-CDR1: SEQ ID NO: 11
VL-CDR2: DAS, and
VL-CDR3: SEQ ID NO: 12
in an amount of from about 5 μg to about 50 mg,
b. acetate buffer and sorbitol, and the pH is about 5.5.

In an embodiment of the unit dosage form the acetate buffer and the sorbitol is comprised in a concentration ratio of between 1:5 and 1:10, such as a ratio of the concentrations of 1:6, 1:7, 1:8 or 1:9.

In a further embodiment the osmolality of the unit dosage form is from about 210 to about 250, such as 220, 230, 240 or 250 mOsm/kg.

In a further embodiment the invention relates to a unit dosage form, comprising
a. a bispecific antibody comprising a first binding region binding to human CD3 which comprises the CDR sequences:
   VH-CDR1: SEQ ID NO: 1
   VH-CDR2: SEQ ID NO: 2
   VH-CDR3: SEQ ID NO: 3
   VL-CDR1: SEQ ID NO: 4
   VL-CDR2: GTN, and
   VL-CDR3: SEQ ID NO: 5,
   and a second binding region binding to human CD20 which comprises the CDR sequences:
   VH-CDR1: SEQ ID NO: 8
   VH-CDR2: SEQ ID NO: 9
   VH-CDR3: SEQ ID NO: 10
   VL-CDR1: SEQ ID NO: 11
   VL-CDR2: DAS, and
   VL-CDR3: SEQ ID NO: 12
   in an amount of from about 5 μg to about 50 mg,
b. acetate buffer at a concentration of about 30 mM,
c. sorbitol at a concentration of about 150 mM, and a pH of about 5.5.

In an embodiment of the above described unit dosage forms the amount of the bispecific antibody is from about 50 μg to about 40 mg, such as from 50 μg to 40 mg.

In an embodiment of the unit dosage form the amount of the bispecific antibody is from about 100 μg to about 30 mg. In another embodiment of the unit dosage form the amount of the bispecific antibody is about 150 μg. In another embodiment of the unit dosage form the amount of the bispecific antibody is about 200 μg. In another embodiment of the unit dosage form the amount of the bispecific antibody is about 250 μg. In another embodiment of the unit dosage form the amount of the bispecific antibody is about 300 μg. In another embodiment of the unit dosage form the amount of the bispecific antibody is about 350 μg. In another embodiment of the unit dosage form the amount of the bispecific antibody is about 400 μg. In another embodiment of the unit dosage form the amount of the bispecific antibody is about 450 μg. In another embodiment of the unit dosage form the amount of the bispecific antibody is about 500 μg. In another embodiment of the unit dosage form the amount of the bispecific antibody is about 600 μg. In another embodiment of the unit dosage form the amount of the bispecific antibody is about 700 μg. In another embodiment of the unit dosage form the amount of the bispecific antibody is about 800 μg. In another embodiment of the unit dosage form the amount of the bispecific antibody is about 900 μg. In another embodiment of the unit dosage form the amount of the bispecific antibody is about 1 mg. In another embodiment of the unit dosage form the amount of the bispecific antibody is about 2 mg. In another embodiment of the unit dosage form the amount of the bispecific antibody is about 3 mg. In another embodiment of the unit dosage form the amount of the bispecific antibody is about 4 mg. In another embodiment of the unit dosage form the amount of the bispecific antibody is about 5 mg. In another embodiment of the unit dosage form the amount of the bispecific antibody is about 6 mg. In another embodiment of the unit dosage form the amount of the bispecific antibody is about 7 mg. In another embodiment of the unit dosage form the amount of the bispecific antibody is about 8 mg. In another embodiment of the unit dosage form the amount of the bispecific antibody is about 9 mg. In another embodiment of the unit dosage form the amount of the bispecific antibody is about 10 mg. In another embodiment of the unit dosage form the amount of the bispecific antibody is about 11 mg. In another embodiment of the unit dosage form the amount of the bispecific antibody is about 12 mg. In another embodiment of the unit dosage form the amount of the bispecific antibody is about 13 mg. In another embodiment of the unit dosage form the amount of the bispecific antibody is about 14 mg. In another embodiment of the unit dosage form the amount of the bispecific antibody is about 15 mg. In another embodiment of the unit dosage form the amount of the bispecific antibody is about 16 mg. In another embodiment of the unit dosage form the amount of the bispecific antibody is about 17 mg. In another embodiment of the unit dosage form the amount of the bispecific antibody is about 18 mg. In another embodiment of the unit dosage form the amount of the bispecific antibody is about 19 mg. In another embodiment of the unit dosage form the amount of the bispecific antibody is about 20 mg. In another embodiment of the unit dosage form the amount of the bispecific antibody is about 21 mg. In another embodiment of the unit dosage form the amount of the bispecific antibody is about 22 mg. In another embodiment of the unit dosage form the amount of the bispecific antibody is about 23 mg. In another embodiment of the unit dosage form the amount of the bispecific antibody is about 24 mg. In another embodiment of the unit dosage form the amount of the bispecific antibody is about 25 mg. In another embodiment of the unit dosage form the amount of the bispecific antibody is about 26 mg. In another embodiment of the unit dosage form the amount of the bispecific antibody is about 27 mg. In another embodiment of the unit dosage form the amount of the bispecific antibody is about 28 mg. In another embodiment of the unit dosage form the amount of the bispecific antibody is about 29 mg such as about 30 mg.

In an embodiment of the unit dosage form the amount of the bispecific antibody is from 100 μg to 30 mg. In another embodiment of the unit dosage form the amount of the bispecific antibody is 150 μg. In another embodiment of the unit dosage form the amount of the bispecific antibody is 200 μg. In another embodiment of the unit dosage form the amount of the bispecific antibody is 250 μg. In another embodiment of the unit dosage form the amount of the bispecific antibody is 300 μg. In another embodiment of the unit dosage form the amount of the bispecific antibody is 350 μg. In another embodiment of the unit dosage form the amount of the bispecific antibody is 400 μg. In another embodiment of the unit dosage form the amount of the bispecific antibody is 450 μg. In another embodiment of the unit dosage form the amount of the bispecific antibody is 500 μg. In another embodiment of the unit dosage form the amount of the bispecific antibody is 600 μg. In another embodiment of the unit dosage form the amount of the bispecific antibody is 700 μg. In another embodiment of the unit dosage form the amount of the bispecific antibody is 800 μg. In another embodiment of the unit dosage form the amount of the bispecific antibody is 900 μg. In another embodiment of the unit dosage form the amount of the bispecific antibody is 1 mg. In another embodiment of the unit dosage form the amount of the bispecific antibody is 2 mg. In another embodiment of the unit dosage form the amount of the bispecific antibody is 3 mg. In another embodiment of the unit dosage form the amount of the bispecific antibody is 4 mg. In another embodiment of the unit dosage form the amount of the bispecific antibody is 5 mg. In another embodiment of the unit dosage form the amount of the bispecific antibody is 6 mg. In another embodiment of the unit dosage form the amount of the bispecific antibody is 7 mg. In another embodiment of the unit dosage form the amount of the bispecific antibody is 8 mg. In another embodiment of the unit dosage form the amount of the bispecific antibody is 9 mg. In another embodiment of the unit dosage form the amount of the bispecific antibody is 10 mg. In another embodiment of the unit dosage form the amount of the bispecific antibody is 11 mg. In another embodiment of the unit dosage form the amount of the bispecific antibody is 12 mg. In another embodiment of the unit dosage form the amount of the bispecific antibody is 13 mg. In another embodiment of the unit dosage form the amount of the bispecific antibody is 14 mg. In another embodiment of the unit dosage form the amount of the bispecific antibody is 15 mg. In another embodiment of the unit dosage form the amount of the bispecific antibody is 16 mg. In another embodiment of the unit dosage form the amount of the bispecific antibody is 17 mg. In another embodiment of the unit dosage form the amount of the bispecific antibody is 18 mg. In another embodiment of the unit dosage form the amount of the bispecific antibody is 19 mg. In another embodiment of the unit dosage form the amount of the bispecific antibody is 20 mg. In another embodiment of the unit dosage form the amount of the bispecific antibody is 21 mg. In another embodiment of the unit dosage form the amount of the bispecific antibody is 22 mg. In another embodiment of the unit dosage form the amount of the bispecific antibody is 23 mg. In another embodiment of the unit dosage form the amount of the bispecific antibody is 24 mg. In another embodiment of the unit dosage form the amount of the bispecific antibody is 25 mg. In another embodiment of the unit dosage form the amount of the bispecific antibody is 26 mg. In another embodiment of the unit dosage form the amount of the bispecific antibody is 27 mg. In another embodiment of the unit dosage form the amount of the bispecific antibody is 28 mg. In another embodiment of the unit dosage form the amount of the bispecific antibody is 29 mg such as 30 mg.

In another embodiment of the unit dosage form of the invention the first binding region of the bispecific antibody binding to human CD3 comprises the VH and VL sequences of SEQ ID: 6 and 7 and the second binding region of the bispecific antibody binding to human CD20 comprises the VH and VL sequences of SEQ ID:13 and 14. In a preferred embodiment of the unit dosage form of the invention the bispecific antibody is the DuoBody-CD3xCD20 as described above.

In another embodiment the total volume of the unit dosage form of the invention is from about 0.3 mL to about 3 mL, such as from 0.3 mL to 3 mL. In another embodiment the total volume of the unit dosage form of the invention is 0.5 mL. In another embodiment the total volume of the unit dosage form of the invention is 0.8 mL. In another embodiment the total volume of the unit dosage form of the invention is 1 mL. In another embodiment the total volume of the unit dosage form of the invention is 1.2 mL. In another embodiment the total volume of the unit dosage form of the invention is 1.5 mL. In another embodiment the total volume of the unit dosage form of the invention is 1.7 mL. In another embodiment the total volume of the unit dosage form of the invention is 2 mL. In another embodiment the total volume of the unit dosage form of the invention is 2.5 mL. Such a unit dosage form is suitable for subcutaneous administration.

In embodiments where the unit dosage form is for I.V. administration the volume is typically larger, such as between 10 mL and 500 mL. In an embodiment the volume of the unit dosage form is 20 mL. In an embodiment the volume of the unit dosage form is 50 mL. In an embodiment the volume of the unit dosage form is 80 mL. In an embodiment the volume of the unit dosage form is 100 mL. In an embodiment the volume of the unit dosage form is 150 mL.

In an embodiment the volume of the unit dosage form is 200 mL. In an embodiment the volume of the unit dosage form is 250 mL. In an embodiment the volume of the unit dosage form is 300 mL. In an embodiment the volume of the unit dosage form is 350 mL. In an embodiment the volume of the unit dosage form is 400 mL. In an embodiment the volume of the unit dosage form is 450 mL. In an embodiment the volume of the unit dosage form is 500 mL.

The unit dosage form may be prepared by diluting the pharmaceutical composition of the invention with a suitable diluent such as e.g. a diluent consisting of the acetate buffer and sorbitol and a pH of 5.5. It is preferred that the diluent has the same concentration of the buffer and sorbitol as in the pharmaceutical composition so that only the concentration of the bispecific antibody is affected by the dilution.

In another embodiment the invention further provides a container or receptacle comprising the unit dosage form described herein.

Further, the invention provides a method of treating cancer in a subject comprising administering to a subject in need thereof the unit dosage form as described herein for a time sufficient to treat the cancer. In one embodiment the invention relates to a method of treating cancer in a subject comprising subcutaneously administering to a subject in need thereof the unit dosage form. In one embodiment the invention relates to a method of treating cancer in a subject comprising intravenously administering to a subject in need thereof the unit dosage form.

In another embodiment the invention relates to the unit dosage form described above for use in the treatment of cancer. In another embodiment the unit dosage form is for subcutaneous administration. In another embodiment the unit dosage form is for intravenous administration.

The present invention also relates to a kit-of-parts comprising:
a. the pharmaceutical composition as described herein
b. a diluent comprising acetate and sorbitol
c. a receptacle for the unit dosage form
d. directions for dilution and/or for use.

It is preferred that the ratio of the concentrations of acetate to sorbitol is equal in the diluent and the pharmaceutical composition.

In one embodiment of the invention the kit-of-parts comprises:
a. the pharmaceutical composition comprising:
   i. 60 mg/mL of the bispecific antibody, such as DuoBody-CD3xCD20
   ii. 30 mM acetate buffer
   iii. 150 mM sorbitol
   iv. pH is 5.5
b. the diluent comprises:
   v. 30 mM acetate buffer
   vi. 150 mM sorbitol
c. a receptacle for the unit dosage form, and
d. directions for dilution and/or for use.

In an embodiment the invention further relates to a method of preparing a pharmaceutical composition as described herein where the method comprises the steps of mixing in water for injection:
a. 60 to 120 mg/mL of a bispecific antibody comprising a first binding region binding to human CD3 which comprises the CDR sequences:
   VH-CDR1: SEQ ID NO: 1
   VH-CDR2: SEQ ID NO: 2
   VH-CDR3: SEQ ID NO:3
   VL-CDR1: SEQ ID NO: 4
   VL-CDR2: GTN, and
   VL-CDR3: SEQ ID NO: 5,
   and a second binding region binding to human CD20 which comprises the CDR sequences:
   VH-CDR1: SEQ ID NO: 8
   VH-CDR2: SEQ ID NO: 9
   VH-CDR3: SEQ ID NO: 10
   VL-CDR1: SEQ ID NO: 11
   VL-CDR2: DAS, and
   VL-CDR3: SEQ ID NO: 12
b. 3.53 mg/mL of sodium acetate trihydrate
c. 0.32 mg/mL of acetic acid
d. 27.3 mg/mL of sorbitol
and adjusting the pH to 5.5 by adding sodium hydroxide.

In one embodiment of the method of preparing the pharmaceutical composition of the invention a. is 60 mg/mL. In another embodiment of the method of preparing the pharmaceutical composition of the invention a. is 70 mg/mL. In another embodiment of the method of preparing the pharmaceutical composition of the invention a. is 80 mg/mL. In another embodiment of the method of preparing the pharmaceutical composition of the invention a. is 90 mg/mL. In another embodiment of the method of preparing the pharmaceutical composition of the invention a. is 100 mg/mL. In another embodiment of the method of preparing the pharmaceutical composition of the invention a. is 110 mg/mL. In another embodiment of the method of preparing the pharmaceutical composition of the invention a. is 120 mg/mL. In another embodiment of the method of preparing the pharmaceutical composition of the invention a. is 150 mg/mL. In another embodiment of the method of preparing the pharmaceutical composition of the invention a. is 200 mg/mL.

The invention further relates to a method of preparing a unit dosage form as described herein, the method comprising the steps of:
a. preparing the pharmaceutical composition by the method of mixing in water for injection:
   a. 60 to 120 mg/mL of a bispecific antibody, such as 60 mg or 120 mg, comprising a first binding region binding to human CD3 which comprises the CDR sequences: VH-CDR1: SEQ ID NO:1, VH-CDR2: SEQ ID NO:2, VH-CDR3: SEQ ID NO:3, VL-CDR1: SEQ ID NO: 4, VL-CDR2: GTN, and VL-CDR3: SEQ ID NO: 5, and a second binding region binding to human CD20 which comprises the CDR sequences: VH-CDR1: SEQ ID NO:8, VH-CDR2: SEQ ID NO:9, VH-CDR3: SEQ ID NO: 10, VL-CDR1: SEQ ID NO: 11, VL-CDR2: DAS, and VL-CDR3: SEQ ID NO: 12
   b. 3.53 mg/mL of sodium acetate trihydrate
   c. 0.32 mg/mL of acetic acid
   d. 27.3 mg/mL of sorbitol, and adjusting the pH to 5.5 by adding sodium hydroxide
b. preparing a diluent in water for injection comprising:
   i. 3.53 mg/mL of sodium acetate trihydrate
   ii. 0.32 mg/mL of acetic acid
   iii. 27.3 mg/mL of sorbitol; and adding sodium hydroxide to adjust pH to 5.5
c. mixing the pharmaceutical composition and the diluent to a desired bispecific antibody concentration of the unit dosage form.

In a further embodiment the invention relates to a pharmaceutical composition or a unit dosage form, which is obtainable by the methods described above.

TABLE 1
Sequences

| SEQ ID NO: | Clone name | Sequence |
|---|---|---|
| SEQ ID NO: 1 | huCD3 VH CDR1 | GFTFNTYA |
| SEQ ID NO: 2 | huCD3 VH CDR2 | IRSKYNNYAT |
| SEQ ID NO: 3 | huCD3 VH CDR3 | VRHGNFGNSYVSWFAY |
| SEQ ID NO: 4 | huCD3 VL CDR1 | TGAVTTSNY |
| | huCD3 VL CDR2 | GTN |
| SEQ ID NO: 5 | huCD3 VL CDR3 | ALWYSNLWV |
| SEQ ID NO: 6 | huCD3 VH1 | EVKLVESGGGLVQPGGSLRLSCAASGFTFNTYAMNWVRQA |
| | | PGKGLEWVARIRSKYNNYATYYADSVKDRFTISRDDSKSS |
| | | LYLQMNNLKTEDTAMYYCVRHGNFGNSYVSWFAYWGQGTL |
| | | VTVSS |
| SEQ ID NO: 7 | huCD3 VL1 | QAVVTQEPSFSVSPGGTVTLTCRSSTGAVTTSNYANWVQQ |
| | | TPGQAFRGLIGGTNKRAPGVPARFSGSLIGDKAALTITGA |
| | | QADDESIYFCALWYSNLWVFGGGTKLTVL |
| SEQ ID NO: 8 | VH CD20-7D8 | GFTFHDYA |
| | CDR1 | |
| SEQ ID NO: 9 | VH CD20-7D8 | ISWNSGTI |
| | CDR2 | |
| SEQ ID NO: 10 | VH CD20-7D8 | AKDIQYGNYYYGMDV |
| | CDR3 | |
| SEQ ID NO: 11 | VL CD20-7D8 | QSVSSY |
| | CDR1 | |
| | VL CD20-7D8 | DAS |
| | CDR2 | |
| SEQ ID NO: 12 | VL CD20-7D8 | QQRSNWPIT |
| | CDR3 | |
| SEQ ID NO: 13 | VH CD20-7D8 | EVQLVESGGGLVQPDRSLRLSCAASGFTFHDYAMHWVRQA |
| | | PGKGLEWVSTISWNSGTIGYADSVKGRFTISRDNAKNSLY |
| | | LQMNSLRAEDTALWCAKDIQYGNYYYGMDVWGQGTTVTVS |
| | | S |
| SEQ ID NO: 14 | VL CD20-7D8 | EIVLTQSPATLSLSPGERATLSCRASQSVSSYLAWYQQKP |
| | | GQAPRLLIYDASNRATGIPARFSGSGSGTDFTLTISSLEP |
| | | EDFAVYYCQQRSNWPITFGQGTRLEIK |
| SEQ ID NO: 15 | 1gG1 heavy chain | ASTKGPSVFPLAPSSKSTSGGTAALGCLVKDYFPEPVTVS |
| | constant region- | WNSGALTSGVHTFPAVLQSSGLYSLSSVVTVPSSSLGTQT |
| | WT | YICNVNHKPSNTKVDKRVEPKSCDKTHTCPPCPAPELLGG |
| | (amino acids | PSVFLFPPKPKDTLMISRTPEVTCVVVDVSHEDPEVKFNW |
| | positions 118-447 | YVDGVEVHNAKTKPREEQYNSTYRVVSVLTVLHQDWLNGK |
| | according to EU | EYKCKVSNKALPAPIEKTISKAKGQPREPQVYTLPPSREE |
| | numbering). | MTKNQVSLTCLVKGFYPSDIAVEWESNGQPENNYKTTPPV |
| | CH3 region | LDSDGSFFLYSKLTVDKSRWQQGNVFSCSVMHEALHNHYT |
| | underlined | QKSLSLSPGK |
| SEQ ID NO: 16 | IgG1-LFLEDA | ASTKGPSVFPLAPSSKSTSGGTAALGCLVKDYFPEPVTVS |
| | heavy chain | WNSGALTSGVHTFPAVLQSSGLYSLSSVVTVPSSSLGTQT |
| | constant region | YICNVNHKPSNTKVDKRVEPKSCDKTHTCPPCPAPEFEGG |
| | (amino acids | PSVFLFPPKPKDTLMISRTPEVTCVVVAVSHEDPEVKFNW |
| | positions 118-447 | YVDGVEVHNAKTKPREEQYNSTRYVVSVLTVLHQDWLNGK |
| | according to EU | EYKCKVSNKALPAPIEKTISKAKGQPREPQVYTLPPSREE |
| | numbering). | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| SEQ ID NO: 17 | IgG1 F405L | ASTKGPSVFPLAPSSKSTSGGTAALGCLVKDYFPEPVTVS |
| | (amino acids | WNSGALTSGVHTFPAVLQSSGLYSLSSVVTVPSSSLGTQT |
| | positions 118-447 | YICNVNHKPSNTKVDKRVEPKSCDKTHTCPPCPAPELLGG |
| | according to EU | PSVFLFPPKPKDTLMISRTPEVTCVVVDVSHEDPEVKFNW |
| | numbering) | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFLLYSKLTVDKSRWQQGNVFSCSVMHEALHNHYT |
| | | QKSLSLSPGK |
| SEQ ID NO: 18 | IgG1-K409R | ASTKGPSVFPLAPSSKSTSGGTAALGCLVKDYFPEPVTVS |
| | (amino acids | WNSGALTSGVHTFPAVLQSSGLYSLSSVVTVPSSSLGTQT |
| | positions 118-447 | YICNVNHKPSNTKVDKRVEPKSCDKTHTCPPCPAPELLGG |
| | according to EU | PSVFLFPPKPKDTLMISRTPEVTCVVVDVSHEDPEVKFNW |
| | numbering) | YVDGVENHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSRLTVDKSRWQQGNVFSCSVMHEALHNHYT |
| | | QKSLSLSPGK |
| SEQ ID NO: 19 | IgG1-LFLEDA- | ASTKGPSVFPLAPSSKSTSGGTAALGCLVKDYFPEPVTVS |
| | F405L (FEAL) | WNSGALTSGVHTFPAVLQSSLGYSLSSVVTVPSSLGTQTY |
| | (amino acids | ICNVNHKPSNTKVDKRVEPKSCDKTHTCPPCPAPEFEGGP |
| | positions 118-447 | SVFLFPPKPKDTLMISRTPEVTCVVVAVSHEDPEVKFNWY |
| | according to EU | VDGVEVHNAKTKPREEQYNSTRYVVSVLTVLHQDWLNGKE |
| | numbering) | YKCKVSNKALPAPIEKTISKAKGQPREPQVYTLPPSREEM |
| | | TKNQVSLTCLVKGFYPSDIAVEWESNGQPENNYKTTPPVL |
| | | DSDGSFLLYSKLTVDKSRWQQGNVFSCSVMHEALHNHYTQ |
| | | KSLSLSPGK |
| SEQ ID NO: 20 | IgG1-LFLEDA- | ASTKGPSVFPLAPSSKSTSGGTAALGCLVKDYFPEPVTVS |
| | F409R (FEAR) | WNSGALTSGVHTFPAVLQSSGLYSLSSVVTVPSSSLGTQT |
| | (amino acids | YICNVNHKPSNTKVDKRVEPKSCDKTHTCPPCPAPEFEGG |
| | positions 118-447 | PSVFLFPPKPKDTLMISRTPEVTCVVVAVSHEDPEVKFNW |
| | according to EU | YVDGVEVHNAKTKPREEQYNSTYRVVSVLTVLHQDWLNGK |
| | numbering) | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSRLTVDKSRWQQGNVFSCSVMHEALHNHYT |
| | | QKSLSLSPGK |
| SEQ ID NO: 21 | IgG1 CH3 region | GQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| SEQ ID NO: 22 | Constant region | GQPKAAPSVTLFPPSSEELQANKATLVCLISDGYPGAVTV |
| | human lambda LC | AWKADSSPVKAGVETTTPSKQSNNKYAASSYLSLTPEQWK |
| | | SHRSYSCQVTHEGSTVEKTVAPTECS |
| SEQ ID NO: 23 | Constant region | RTVAAPSVFIFPPSDEQLKSGTASVVCLLNNFYPREAKVQ |
| | human kappa LC | WKVDNALQSGNSQESVTEQDSKDSTYSLSSTLTLSKADYE |
| | | KHKVYACEVTHQGLSSPVTKSFNRGEC |
| SEQ ID NO: 24 | huCD3 light chain | QAVVTQEPSFSVSPGGTVTLTCRSSTGAVTTSNYANWVQQ |
| | VL + CL | TPGQAFRGLIGGTNKRAPGVPARFSGSLIGDKAALTITGA |
| | | QADDESIYFCALWYSNLWVFGGGTKLTVLGQPKAAPSVTL |
| | | FPPSSEELQANKATLVCLISDFYPGAVTVAWKADSSPVKA |
| | | GVETTTPSKQSNNKYAASSYLSLTPEQWKSHRSYSCQVTH |
| | | EGSTVEKTVAPTECS |
| SEQ ID NO: 25 | CD20-7D8 light | EIVLTQSPATLSLSPGERATLSCRASQSVSSYLAWYQQKP |
| | chain VL + CL | GQAPRLLIYDASNRATGIPARFSGSGSGTDFTLTISSLEP |
| | | EDFAVYYCQQRSNWPITFGQGTRLEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |

### EXAMPLES

The pharmaceutical composition of the invention may be prepared by mixing the ingredients as listed in table 2.

TABLE 2
Composition of a DuoBody-CD3xCD20 pharmaceutical composition
of the invention.

| | Quantity | | | Reference |
|---|---|---|---|---|
| Ingredients | per mL | | Function | to Grade |
| Active ingredient | | | | |
| DuoBody-CD3xCD20 | 5.0 | mg | Active ingredient | |
| Inactive Ingredients | | | | |
| Sodium acetate | 3.53 | mg | Buffering agent | USP/Ph.Eur. |
| trihydrate | | | | |
| Acetic acid | 0.32 | mg | Buffering agent | NF/Ph.Eur. |
| Sodium hydroxide | q.s | | pH adjustment | Ph.Eur. |
| Sorbitol | 27.3 | mg | Tonicifier | NF/Ph.Eur. |
| Water for injection | q.s to 1.00 | mL | Solvent | USP/Ph.Eur. |

### Example 1: Stability of Duobody-CD3xCD200 in Different Formulations

### Abbreviations

| Abbreviation/Term | Definition |
|---|---|
| A₂₈₀ | Absorbance at 280 nm |
| A₅₅₀ | Absorbance at 550 nm |
| BCM | Barycentric Mean |
| CE | Capillary Electrophoresis |
| DSF | Differential Scanning Fluorimetry |
| DLS | Dynamic Light Scattering |
| HC | Heavy Chain |
| HMW | High Molecular Weight |
| HPLC | High Performance Liquid Chromatography |
| icIEF | Imaged Capillary Isoelectric Focusing |
| LC | Light Chain |
| LMW | Low Molecular Weight |
| NaCl | Sodium Chloride |
| NGHC | non-glycosylated heavy chain |
| RH | Relative Humidity |
| Ppm | Parts per million |
| % Pd | Percent Polydispersity |
| SDS | Sodium Dodecyl Sulfate |
| SEC | Size Exclusion Chromatography |
| SLS | Static Light Scattering |
| T_{agg} | Onset of Aggregation Temperature |
| Tₒₙₛₑₜ | Onset of Unfolding Temperature |
| Tₘ | Melting Temperature (=Midpoint of unfolding) |
| UV | Ultraviolet |

Material

Duobody-CD3xCD2 was formulated at 2 mg/mL, unless stated otherwise.

Methods

Thermal Stability by Fluorescence/Static Light Scattering

Conformational and Colloidal stability was determined by a combined Fluorescence/Static Light Scattering (SLS) measurement on the UNit instrument (Unchained Labs). The measurement utilizes increasing thermal stress to induce protein unfolding and aggregation to assess conformational and colloidal stability. The unfolded state transitions caused by increased thermal stress are detected by changes in intrinsic fluorescence of the Trp (and Tyr) residues of the protein due to changes in the local environment upon protein unfolding. As buried tryptophan residues are exposed, the maximum emission wavelength moves to longer wavelengths. The barycentric mean (BCM), the wavelength at which the fluorescence emission spectrum is equally divided, is plotted, showing the conformational change of the protein over temperature. The fluorescence analysis provides the onset of unfolding temperature (Tₒₙₛₑₜ) and the melting temperature (Tₘ) values, both of which are generated from the BCM curves. The Tₒₙₛₑₜ provides the calculated temperature at which the protein begins to unfold. The Tₘ value is a transition midpoint of the protein from the folded state to the unfolded state.

The UNit measurement also provided SLS measurements for determining protein colloidal stability. The sample was illuminated by laser light which is scattered by the molecules in solution. The intensity of static light scattering is proportional to the average molecular weight of species in solution. This analysis is therefore sensitive to protein aggregation over the temperature ramp. The static light scattering was measured at 266 nm, to detect smaller aggregates, as well as at 473 nm, for the detection of larger aggregate species. The onset of aggregation temperature (T_{agg}) was determined from these data, which is the temperature at which the protein begins to aggregate. These data are best analyzed by large changes in count intensity—higher counts indicate more light has been scattered due to the formation of protein aggregates. Over an increasing temperature ramp, changes in SLS count data in the 10³ scale is typically attributed to significant protein aggregation, minimal changes in SLS counts are attributed to partial aggregation, and no change in SLS counts over the temperature ramp is indicative of negligible protein aggregation.

Appearance

Appearance was determined by visual evaluation.

pH

pH was measured using a Mettler Toledo SevenMulti pH meter.

Viscosity

Viscosity was measured using a Wells-Brookfield Cone/Plate Rheometer.

Osmolality

Osmolality was measured using an osmometer.

Protein Concentration by Absorbance A₂₈₀

Protein concentration was determined by UV/Vis Spectroscopy (absorbance measurement at 280 nm (A280) using an Agilent UV/Vis Spectrophotometer (Model 8453)

Size Exclusion Chromatography (SEC)

Size exclusion chromatography was performed on an Agilent 1100 and 1200 HPLC system, using a TOSOH, TSK-gel G3000SWxL (7.8×300 mm) column (Sigma, cat. no. 08541).

Imaged Capillary Isoelectric Focusing (icIEF)

Imaged capillary isoelectric focusing was performed using an iCE 3 Analyzer equipped with PrinCE Autosampler.

Reduced and Non-Reduced Microchip Capillary Electrophoresis—Sodium Dodecyl Sulfate

Microchip capillary electrophoresis (both reduced and non-reduced) was performed using a Labchip GXII instrument according to manufacturer's instructions.

Dynamic Light Scattering (DLS)

Dynamic light scattering analysis was performed using a Wyatt DynaPro Plate Reader. DLS analysis assessed protein size and aggregation at room temperature. For DLS analysis, time autocorrelation functions of scattered light are determined, and an average size of the molecules in solution is calculated based on a single exponential cumulant fitting of data. The reportable values are polydispersity and hydrodynamic radius. For protein samples comprised of a single distribution of monomer, the sample is considered monodisperse; however, for samples containing multiple particle size populations, the samples are considered polydisperse. The percentage polydispersity index (% Pd) is a measure of the width of the particle size distribution—the higher the % Pd, the wider the distribution of particles. Therefore, samples with high % Pd are typically found to contain (large) aggregates. The hydrodynamic radius of a non-spherical protein particle is the radius of a sphere that has the same translational diffusion speed as the particle. The diffusion speed depends on the molecular weight of the particle, the surface structure, as well as the concentration and type of ions in the formulation. A larger hydrodynamic radius in a monodisperse size distribution can be attributed to the presence of higher order oligomers (e.g. tetramers) in solution, but not large aggregates.

Solubility Screening

To determine the solubility of Duobody-CD3xCD20, the material was first formulated in the selected buffers at a low start concentration using centrifugal concentrators. Consecutively, the solution was concentrated by timed spins of 20, 50, 60 and 90 min to a target concentration of >120 mg/mL. The protein concentration was measured after each spin.

Results

1. Baseline Biophysical and Excipient Screening

Initial biophysical screening was performed to select buffer/pH/excipients combinations to move forward into a more detailed screening. Baseline biophysical and excipient screening involved thermal stability screening of the DuoBody-CD3xCD20 (2 mg/mL), in a wide range of buffer/pH/excipient combinations by Fluorescence/SLS and DLS. A list of buffers and their pH values used for the initial screen are listed in Table 3.

Table 1 displays the data obtained from the initial buffer screen, wherein 30 mM acetate and 30 mM histidine buffers were tested either with or without excipients (150 mM NaCl, 150 mM arginine, 150 mM sorbitol or 150 mM sucrose). Fluorescence/SLS measurements were used to assess thermal stability and DLS to determine aggregation of Duobody-CD3xCD20 (2 mg/mL) at room temperature. Fluorescence/SLS analysis provided the melting temperature (Tₘ), onset of unfolding (Tₒₙₛₑₜ) and T_{agg}. DLS analysis provided information on polydispersity and hydrodynamic radius of the protein.

In the thermal stability analysis, the Tₒₙₛₑₜ and Tₘ are slightly higher in the acetate formulations (ranging from 53-58° C. and 60-62.5° C., respectively) when compared to the corresponding histidine formulations (ranging from 53-55° C. and 59-61° C., respectively). Higher Tₒₙₛₑₜ and Tₘ values are indicative of better thermal stability of the protein. Differences in Tₒₙₛₑₜ and Tₘ between the different excipients are low but may point to a slightly lower stability in the presence of arginine and slightly higher stability with sorbitol or sucrose. T_{agg} determination by SLS showed that for both acetate and histidine formulations, the addition of NaCl or arginine resulted in a lower T_{agg} (59-60° C.) compared to the formulation with sorbitol or sucrose or without excipient. Partial aggregation at 66° C. was observed in acetate formulations with sorbitol or sucrose, whereas no aggregation was observed in the histidine buffer with these excipients.

DLS at room temperature showed a negative effect on the aggregation behavior of the molecule in the presence of sucrose, as exemplified by a larger average radius and a multimodal consistency. Also sorbitol seems to induce a small increase in average radius and % Pd.

Based on the data obtained from the initial screening, it was concluded that DuoBody-CD3xCD20 is stable and monodisperse in acetate pH 5.5, histidine pH 6.0 and histidine pH 6.5 buffers without excipients. Sorbitol and sucrose increased thermal stability slightly. NaCl and arginine decreased thermal stability. Based on the DLS results in the initial screening, sucrose was deselected as excipient for further solubility screening. Acetate pH 5.5, histidine pH 6.0 and histidine pH 6.5 formulations with or without excipients (150 mM NaCl, 150 mM arginine or 150 mM sorbitol) were selected for further solubility studies.

TABLE 3
Results from initial baseline biophysical and excipient screen of Duobody-
CD3xCD20 (2 mg/mL) in indicated formulations.
Thermal scan

| | Tₒₙₛₑₜ | Tₘ | |
|---|---|---|---|
| Formulation (Buffer, pH | (Fluores- | (Fluores- | |
| excipients | cence) | cence) | T_{agg} 266 nm (SLS) |
| Acetate pH 5.0 | 57 | 61.0 | No Aggregation |
| Acetate pH 5.5 | 58 | 62.0 | Partial Aggregation (66) |
| Acetate pH 5.5 NaCl | 57 | 61.5 | 60 |
| Acetate pH 5.5 Arginine | 53 | 60.0 | 60 |
| Acetate pH 5.5 Sorbitol | 58 | 62.5 | Partial Aggregation (66) |
| Acetate pH 5.5 Sucrose | 58 | 62.5 | Partial Aggregation (66) |
| Histidine pH 5.5 | 53 | 59.0 | No Aggregation |
| Histidine pH 6.0 | 55 | 60.0 | No Aggregation |
| Histidine pH 6.0 NaCl | 53 | 60.0 | 59 |
| Histidine pH 6.0 Arginine | 53 | 59.0 | 60 |
| Histidine pH 6.0 Sorbitol | 53 | 61.0 | No Aggregation |
| Histidine pH 6.0 Sucrose | 55 | 61.0 | No Aggregation |
| Histidine pH 6.5 | 55 | 61.0 | No Aggregation |

DLS

| Formulation (Buffer, pH | Radius | |
|---|---|---|
| excipients | (nm) | % Pd |
| Acetate pH 5.0 | 5.009 | 5.7 |
| | 5.007 | 5.1 |
| Acetate pH 5.5 | 5.167 | 8.7 |
| | 5.337 | 18.4 |
| Acetate pH 5.5 NaCl | 5.179 | 3.7 |
| | 5.158 | 2.4 |
| Acetate pH 5.5 Arginine | 5.327 | 9.6 |
| | 5.398 | 12 |
| Acetate pH 5.5 Sorbitol | 7.927 | Multimodal |
| | 7.787 | Multimodal |
| Acetate pH 5.5 Sucrose | 32.649 | Multimodal |
| | 33.18 | Multimodal |
| Histidine pH 5.5 | 5.032 | 4.2 |
| | 5.016 | 6.3 |
| Histidine pH 6.0 | Correlation fit failed | |
| | 5.19 | 7.9 |
| Histidine pH 6.0 NaCl | 5.219 | 6.6 |
| | 5.363 | 14.2 |
| Histidine pH 6.0 Arginine | 5.288 | 8.3 |
| | 5.503 | 14.7 |
| Histidine pH 6.0 Sorbitol | 5.801 | 24.6 |
| | 6.228 | 23.7 |
| Histidine pH 6.0 Sucrose | 30.305 | Multimodal |
| | 36.799 | Multimodal |
| Histidine pH 6.5 | 5.142 | 9.6 |
| | 5.263 | 21 |

2. Solubility Screening

The second stage of the baseline biophysical screening study involved solubility screening of pH/buffer combinations selected from the initial baseline biophysical screening combined with excipients. A list of the buffers used in the second screening study can be found in Table 4.

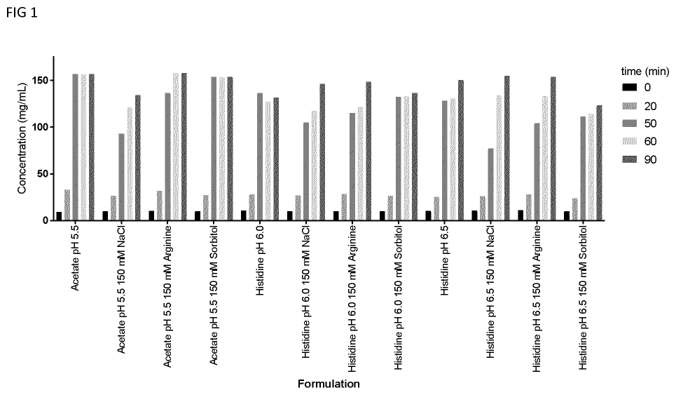

To determine the solubility in the presence of excipients, the material was formulated in the selected buffers and consecutively concentrated using centrifugal concentrators with timed spin intervals. FIG. 1 shows the concentration of each formulation after the spin intervals of 20, 50, 60 and 90 min. Acetate formulations with and without sorbitol concentrated to 150 mg/mL fastest (50 min). Histidine formulations with and without sorbitol concentrated fast (50 min), but to a lower concentration (120 mg/mL). All other formulations enabled concentrating to 120 mg/mL, albeit this concentration was reached slower (60-90 min) compared to the acetate formulations.

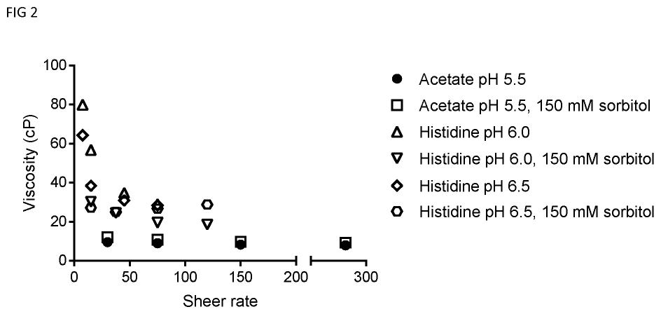

The concentrated samples (at their final concentration of 120-150 mg/mL) were further analyzed for Fluorescence/SLS and DLS (Table 4) and viscosity (FIG. 2).

Table 4 shows that formulations without excipients and with sorbitol had highest Tₘ. T_{agg} values in formulations without excipient and with sorbitol were difficult to interpret since transitions were unsharp compared to measurements in formulations with NaCl and Arg.

DLS data at RT show a general increase in % Pd for the higher DuoBody-CD3xCD20 concentration compared to the low concentration in Table 3 (>15% is interpreted as polydisperse). The variation in average hydrodynamic radius may be influenced by the viscosity of the concentrated material, which hampers a proper ranking of the formulations.

FIG. 2 shows the viscosity (cP) of the different formulations with and without sorbitol. Acetate formulations showed a viscosity ranging from 7.9-12.1 cP. By contrast, the histidine formulations without sorbitol were more viscous than acetate formulations (ranging from 28.4-79.9 cP). Addition of sorbitol decreased the viscosity of the histidine formulations (ranging from 18-30 cP), while sorbitol had no effect on the viscosity of the acetate formulations.

TABLE 4
Results from concentrated samples (Duobody-CD3xCD20
[120-150 mg/mL] in indicated formulations).
Thermal scan

| | | | SLS | SLS |
|---|---|---|---|---|
| | | | 266 | 473 |
| | Fluorescence | | nm | nm |
| Formulation | Tₒₙₛₑₜ | Tₘ | T_{agg} | T_{agg} |
| Acetate pH 5.5 | 58.7 | 62.7 | 59.0 | 59.5 |
| Acetate pH 5.5 150 mM NaCl | 57.1 | 59 | 57.7 | 57.8 |
| Acetate pH 5.5 150 mM | 56.6 | 59 | 57.3 | 57.7 |
| Arginine | | | | |
| Acetate pH 5.5 150 mM Sorbitol | 58.3 | 59.9 | 58.4 | 59.5 |
| Histidine pH 6.0 | 56.4 | 58.1 | 58.8 | 57.4 |
| Histidine pH 6.0 150 mM NaCl | 56.4 | 57.0 | 57.1 | 57.3 |
| Histidine pH 6.0 150 mM | 56.2 | 57.1 | 57.2 | 57.4 |
| Arginine | | | | |
| Histidine pH 6.0 150 mM | 56.8 | 58.4 | 56.6 | 57.0 |
| Sorbitol | | | | |
| Histidine pH 6.5 | 56.9 | 58.6 | 55.1 | 58.0 |
| Histidine pH 6.5 150 mM NaCl | 56.7 | 57.5 | 57.7 | 57.7 |
| Histidine pH 6.5 150 mM | 56.7 | 57.5 | 57.5 | 57.5 |
| Arginine | | | | |
| Histidine pH 6.5 150 mM | 57 | 58.9 | 58.1 | 58.5 |
| Sorbitol | | | | |

DLS

| | | | | |
|---|---|---|---|---|
| | | | Radius | |
| Buffer | pH | Excipient | (nm) | % Pd |

| | | |
|---|---|---|
| Acetate pH 5.5 | 3.73 | 21.6 |
| | 3.76 | 23 |
| Acetate pH 5.5 150 mM NaCl | 8.31 | 16.9 |
| | 8.31 | 18.1 |
| Acetate pH 5.5 150 mM Arginine | 7.1 | 19.1 |
| | 6.91 | 17.3 |
| Acetate pH 5.5 150 mM Sorbitol | 6.43 | Multimodal |
| | 6.57 | Multimodal |
| Histidine pH 6.0 | 4.72 | 23.9 |
| | 4.67 | 23.9 |
| Histidine pH 6.0 150 mM NaCl | 8.6 | 19.6 |
| | 8.56 | 19.9 |
| Histidine pH 6.0 150 mM | 8.06 | 23.9 |
| Arginine | 7.92 | 23.9 |
| Histidine pH 6.0 150 mM Sorbitol | 5.67 | Multimodal |
| | 5.55 | Multimodal |
| Histidine pH 6.5 | 4.95 | 48.3 |
| | 4.56 | 23.6 |
| Histidine pH 6.5 150 mM NaCl | 10.6 | 46.2 |
| | 10.7 | 34.6 |
| Histidine pH 6.5 150 mM | — | — |
| Arginine | — | — |
| Histidine pH 6.5 150 mM Sorbitol | 26.3 | Multimodal |
| | — | — |

Furthermore, osmolality of the concentrated samples in acetate pH 5.5 with or without sorbitol, histidine buffer pH 6.0 with sorbitol and histidine pH 6.5 with sorbitol was measured using an osmometer. Results are shown in Table 5.

The osmolality of acetate pH 5.5 without sorbitol ranged from 70-80 mOsm/kg. The osmolality of the acetate and histidine formulations with sorbitol ranged from 220-230 mOsm/kg, which is closer to the osmolality of normal plasma (275-295 mOsm/kg; Rasouli 2016 Clin Biochem 49 (12):936-41).

TABLE 5
Osmolality of Duobody-CD3xCD20 (120-150 mg/mL) in indicated
formulations

| | Osmolality |
|---|---|
| Formulation | (mOsm/kg) |
| Acetate pH 5.5 | 70-80 |
| Acetate pH 5.5 150 mM | 220-230 |
| Sorbitol | |
| Histidine pH 6.0 150 mM | 220-230 |
| Sorbitol | |
| Histidine pH 6.5 150 mM | 220-230 |
| Sorbitol | |

Based on the above results 30 mM acetate pH 5.5 with 150 mM sorbitol was found to be a favorable formulation of Duobody-CD3xCD20, as acetate with sorbitol showed comparable thermal stability with histidine formulations, while it supported the solubility at high concentrations (150 mg/mL) most efficiently and it was the least viscous formulation.

3. Real Time and Accelerated Stability

Real time stability of Duobody-CD3xCD20 in 30 mM acetate, 150 mM sorbitol, pH 5.5 was evaluated using assays described (appearance, pH, UV [A₂₈₀], SEC, icIEF, CE-SDS [reduced and non-reduced]) at different time points ranging from 0-12 months.

Table 6 shows the stability test results of Duobody-CD3xCD20 (5 mg/mL) samples that were stored at 5±3° C. for 0, 2, 3 or 6 months.

Table 7 shows the results of the stability tests of Duobody-CD3xCD20 (5 mg/mL) samples that were stored at 25±° C. for 0, 1, 2, 3 or 6 months.

Table 8 shows the stability test results of Duobody-CD3xCD20 (60 mg/mL) samples that were stored at 5±3° C. for 0, 2, 3, 6, 9 or 12 months.

Table 9 shows the results of the stability tests of Duobody-CD3xCD20 (60 mg/mL) samples that were stored at 25±3° C. for 0, 1, 2, 3 or 6 months.

After 12 and 6 months storage at 5±3° C. and 25±3° C. respectively, all samples remained stable by all test methods at a concentration of 5 mg/mL and 60 mg/mL. The samples stored at 53° C. showed no significant changes by any test methods at the 6 month or 12 month time point compared to the study start. Expected minor changes in the purity profile during accelerated stability testing at 25±3° C. were observed by UV spectrometry, cIEF, reduced CE-SIDS, and SEC testing.

TABLE 6
Example data from stability tests of Duobody-CD3xCD20 (5 mg/mL) at
5 ± 3° C.

| | Time Point (months) | | | |
|---|---|---|---|---|
| Assay | 0 | 2 | 3 | 6 |
| pH | 5.5 | 5.3 | 5.4 | 5.3 |
| UV Spectrophotometry | 4.9 mg/mL | 5.1 mg/mL | 5.1 mg/mL | 5.3 mg/mL |
| (mg/mL) | | | | |
| CE-SDS Non-Reduced | 98.2% | 96.1% | 95.5% | 97.3% |
| % Main Peak | | | | |
| CE-SDS Reduced % HC + LC | 98.4% | 96.7% | 96.7% | 95.9% |
| cIEF | CR | CR | CR | CR |
| cIEF neutral peak pI | 8.8 | 8.7 | 8.7 | 8.8 |
| cIEF % acidic peaks | 63.6% | 61.1% | 62.8% | 62.1% |
| cIEF % main peak | 34.4% | 37.2% | 35.6% | 36.1% |
| cIEF % basic peaks | 2.0% | 1.7% | 1.6% | 1.8% |
| SEC-UPLC % Main Peak | 97.4% | 97.3% | 97.6% | 97.8% |
| SEC-UPLC % HMW | 2.5% | 2.2% | 2.0% | 2.1% |
| SEC-UPLC % LMW | 0.2% | 0.4% | 0.5% | 0.1% |
| Appearance | Colorless, | Colorless, clear | Colorless, | Colorless, |
| Assay | Time Point (months) | | | |
| | clear | solution, | clear, | clear |
| | solution, no | contains a few | solution, free | solution, free |
| | visible | visible thread- | from visible | from visible |
| | particulates | like particulates | particulates | particulates |

TABLE 7
Example data from stability tests of Duobody-CD3xCD20 (5 mg/mL) at
25 ± 3° C.

| | Time point (months) | | | | |
|---|---|---|---|---|---|
| Assay | 0 | 1 | 2 | 3 | 6 |
| pH | 5.5 | 5.4 | 5.4 | 5.4 | 5.4 |
| UV Spectrophotometry | 4.9 mg/mL | 5.2 mg/mL | 5.6 mg/mL | 6.0 mg/mL | 7.4 mg/mL |
| (mg/mL) | | | | | |
| CE-SDS Non-Reduced % Main | 98.2% | 95.7% | 95.2% | 94.7% | 95.9% |
| Peak | | | | | |
| CE-SDS Reduced % HC + LC | 98.4% | 96.1% | 95.1% | 94.6% | 92.7% |
| cIEF | CR | CR | CR | CR | CR |
| cIEF neutral peak pI | 8.8 | 8.7 | 8.7 | 8.7 | 8.7 |
| cIEF % acidic peaks | 63.6% | 64.4% | 65.6% | 68.7% | 74.7% |
| cIEF % main peak | 34.4% | 33.8% | 32.4% | 29.2% | 23.2% |
| cIEF % basic peaks | 2.0% | 1.7% | 2.0% | 2.2% | 2.2% |
| SEC-UPLC % Main Peak | 97.4% | 97.6% | 97.9% | 97.0% | 96.7% |
| SEC-UPLC % HMW | 2.5% | 1.9% | 1.9% | 2.4% | 2.7% |
| SEC-UPLC % LMW | 0.2% | 0.4% | 0.2% | 0.6% | 0.5% |
| Assay | Time point months | | | | |
| Appearance | Colorless, | Slightly | Colorless, | Colorless, | Colorless, |
| | clear | yellow, | clear | clear | clear |
| | solution, | clear | solution, | solution, | solution free |
| | no visible | solution, | contains a | free from | from few |
| | particulates | free from | few visible | visible | visible |
| | | visible | thread-like | particulates | particulates |
| | | particulates | particulates | | |

TABLE 8
Example data from stability tests of Duobody-CD3×CD20 (60 mg/mL) at 5 ± 3° C.

| | Time point (months) | | | | | |
|---|---|---|---|---|---|---|
| Assay | 0 | 2 | 3 | 6 | 9 | 12 |
| pH | 5.51 | 5.4 | 5.5 | 5.4 | 5.4 | 5.4 |
| UV | 61.5 | 65.1 | 66.0 | 66.4 | 69.1 | 71.4 |
| Spectrophotometry | mg/mL | mg/mL | mg/mL | mg/mL | mg/mL | mg/mL |
| (mg/mL) | | | | | | |
| CE-SDS Reduced % HC | 64.9% | 64.9% | 65.1% | 64.3% | 63.5% | 64.3% |
| CE-SDS Reduced % LC | 31.9% | 31.6% | 31.7% | 31.6% | 32.4% | 31.5% |
| CE-SDS Reduced % NGHC | 0.9% | 0.7% | 0.6% | 0.7% | 0.8% | 0.8% |
| CE-SDS Non-Reduced | 98.3% | 95.8% | 95.4% | 96.4% | 97.1% | 97.0% |
| % Main Peak | | | | | | |
| Assay | Time point (months) | | | | | |
| SEC-UPLC % Main Peak | 98.1% | 96.7% | 96.3% | 96.5% | 96.1% | 95.9% |
| SEC-UPLC % HMW | 1.7% | 3.1% | 3.3% | 3.5% | 3.7% | 3.8% |
| SEC-UPLC % LMW | 0.2% | 0.2% | 0.5% | 0.1% | 0.2% | 0.3% |
| cIEF % Main Peak | 33.9% | 36.1% | 35.9% | 35.5% | 35.9% | 34.2% |
| cIEF % Acidic Peaks | 64.0% | 62.3% | 62.4% | 63.0% | 62.2% | 63.5% |
| cIEF % Basic Peaks | 2.1% | 1.6% | 1.7% | 1.7% | 1.9% | 2.3% |
| Appearance | Slightly | Slightly | Slightly | Slightly | Slightly | Slightly |
| | yellow, | yellow, | yellow, | yellow, | yellow, | yellow, |
| | clear | clear | clear | clear | clear and | clear and |
| | solution, no | solution, | solution, | solution, | contains a | free of |
| | visible | contains a | free from | free from | few visible | visible |
| | solids; | few visible | visible | visible | fibrous | particulates |
| | | thread-like | particulates | particulates | particulates | |
| | | particulates | | | | |

TABLE 9
Example data from stability tests of Duobody-CD3xCD20 (60 mg/mL) at
25 ± 3° C.

| | Timepoint (months) | | | | |
|---|---|---|---|---|---|
| Assay | 0 | 1 | 2 | 3 | 6 |
| pH | 5.5 | 5.5 | 5.5 | 5.5 | 5.5 |
| UV | 61.5 mg/mL | 65.8 | 71.7 | 75.9 | 93.3 |
| Spectrophotometry | | | | | |
| Assay | Timepoint (months) | | | | |
| (mg/mL) | | mg/mL | mg/mL | mg/mL | mg/mL |
| CE-SDS Reduced % | 64.9% | 64.7% | 63.8% | 63.1% | 61.6% |
| HC | | | | | |
| CE-SDS Reduced % | 31.9% | 31.7% | 31.8% | 32.0% | 32.1% |
| LC | | | | | |
| CE-SDS Reduced % | 0.9% | 0.8% | 0.9% | 1.1% | 1.3% |
| NGHC | | | | | |
| CE-SDS Non-Reduced | 98.3% | 95.4% | 94.9% | 94.2% | 93.2% |
| % Main Peak | | | | | |
| SEC-UPLC % Main | 98.1% | 95.7% | 95.5% | 94.7% | 93.8% |
| Peak | | | | | |
| SEC-UPLC % HMW | 1.7% | 3.7% | 4.1% | 4.6% | 5.7% |
| SEC-UPLC % LMW | 0.2% | 0.6% | 0.5% | 0.7% | 0.5% |
| cIEF % Main Peak | 33.9% | 35.7% | 31.9% | 31.2% | 26.2% |
| cIEF % Acidic Peaks | 64.0% | 62.7% | 66.0% | 66.6% | 71.5% |
| cIEF % Basic Peaks | 2.1% | 1.6% | 2.1% | 2.1% | 2.3% |
| Appearance | Slightly | Colorless, | Slightly | Slightly | Slightly |
| | yellow, clear | clear | yellow, clear | yellow, clear | yellow, clear |
| | solution, free | solution, | solution, | solution, | solution, |
| | from visible | contains | contains a | free from | free from |
| | particulates | white | few visible | visible | visible |
| | | threadlike | thread-like | particulates | particulates |
| | | particulates | particulates | | |

4. Conclusions

Based upon the results obtained from analytical testing of Duobody-CD3xCD20 in the various formulations, 30 mM acetate, 150 mM sorbitol, pH 5.5 was the optimal formulation for this molecule. This formulation supports concentrations ranging from 2-150 mg/mL. We showed that the DuoBody-CD3xCD20 is stable at 5 and 60 mg/mL (in 30 mM acetate, 150 mM sorbitol, pH 5.5) for up to 12 months at 5±3° C. Furthermore, upon accelerated stability testing at 25±3° C. only minor expected changes were observed for the DuoBody-CD3xCD20 at 5 and 60 mg/mL (in 30 mM acetate, 150 mM sorbitol, pH 5.5) for up to 6 months.

### Example 2: Cytokine Analysis in Blood of Cynomolgus Monkeys Treated with DuoBody-CD3xCD20 Via Intravenous (IV) and Subcutaneous (SC) Routes of Administration

Blood samples were collected from animals in a dose-range finding (DRF) study of DuoBody-CD3xCD20 in female cynomolgus monkeys, and a GLP toxicology study of DuoBody-CD3xCD20 in female and male cynomolgus monkeys at t=0 (pre-dose), 2, 4, 6, 12 and 24 hours. Samples (0.25 mL) were transferred into tubes containing K₂EDTA and processed for obtaining plasma by centrifugation at 3000 rpm (approximately 1500 g) for 10 minutes at 4° C. Plasma was transferred to clear 0.5 mL polypropylene tubes and stored at −80° C. until analysis.

The samples were analyzed according to the manufacturers' protocol using the Milliplex MAP NHP Cytokine Magnetic Bead Panel (Millipore Cat. No. PRCYTOMAG-40K) for use with a BioPlex 200 reader (BioRad) to measure the concentration of IL-1p, IL-2, IL-6, IL-4, IL-8, IL-10, IL-12p40, IL-15, IFNγ, TNFα and MCP-1.

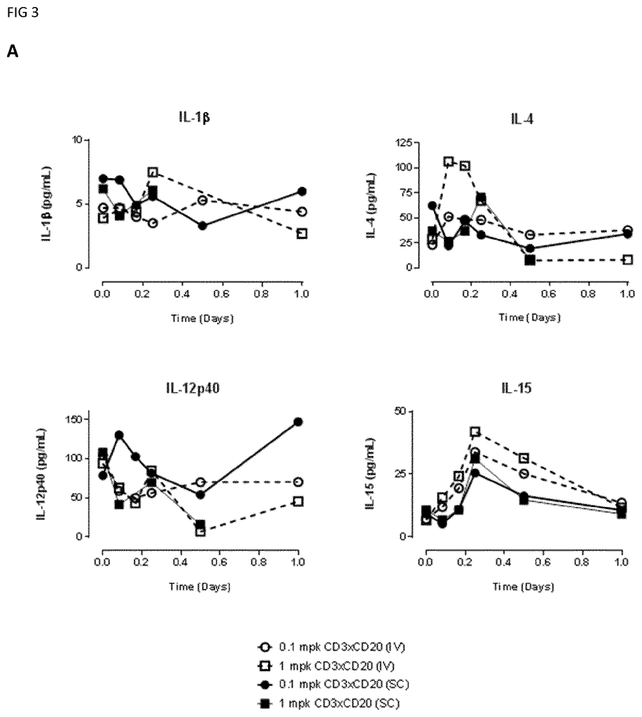

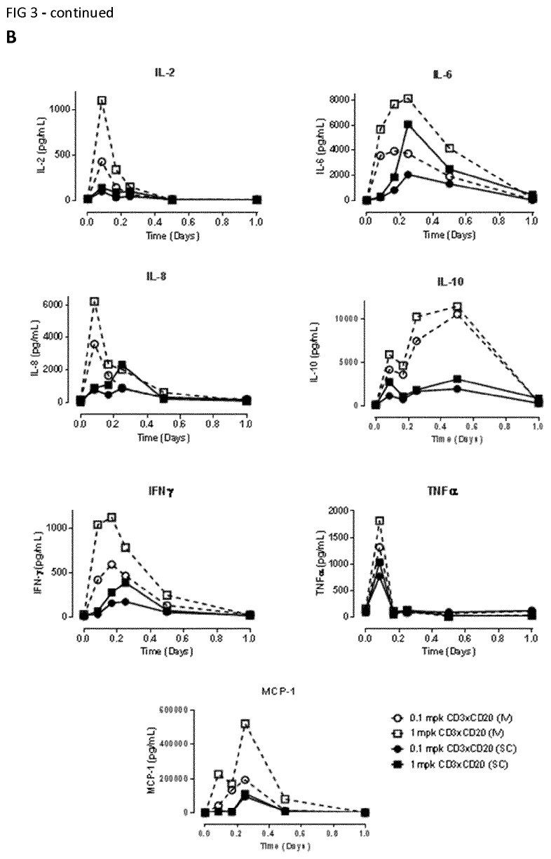

FIG. 3 shows the mean cytokine levels per group in blood from animals which received either a single IV dose (0.1 or 1 mg/kg) or a single SC dose (0.1 or 1 mg/kg) of DuoBody-CD3xCD20 in a pharmaceutical composition of the invention in the GLP toxicology study.

Dosing of DuoBody-CD3xCD20 induced only low levels (below 150 μg/mL) of the cytokines IL-1β, IL-4, IL-12p40 and IL-15 (FIG. 3A).

The cytokines IL-2, IL-6, IL-8, IL-10, IFNγ, TNFα and MCP-1 were more clearly induced upon IV dosing of DuoBody-CD3xCD20, reaching a peak within 2-12 hours after dosing (FIG. 3B). Thereafter cytokine levels returned to base-line. For each of these cytokines, peak levels were lower in the blood of animals which received SC dosing (0.1 or 1 mg/kg) versus the corresponding IV dose levels. For IL-8 and IFN-γ peak levels were both reduced and delayed upon SC dosing compared to IV dosing.

Comparable findings were seen in the DRF study, with the exception that in this (smaller) study there was no difference in IFNγ levels between IV or SC dosed animals.

### Example 3: Evaluation of B Cell Depletion in Cynomolgus Monkeys Following 4× Repeat Dose IV Infusions, a Single IV Dose with Priming Dose, or a Single Dose SC Injection of DuoBody-CD3xCD2 (Dose-Range Finding Study)

Female cynomolgus monkeys received DuoBody-CD3xCD2 in formulations of the invention (30 mM acetate, 150 mM sorbitol, pH 5.5) either via 4 weekly IV infusions (0.01, 0.1 or 1 mg/kg), an IV priming dose (0.01 mg/kg) followed by a IV target dose of 1 mg/kg, or a single SC injection (0.01, 0.1, 1, 10 or 20 mg/kg), as per this overview:

| | | | | Dose | | |
|---|---|---|---|---|---|---|
| | Route | Dose | Dose | Concen- | | Number |
| Group | of | Level | Volume | tration | Days of | of |
| No. | Dosing | (mg/kg) | (mL/kg) | (mg/mL) | Dosing | Females |
| 1 | IV | 0.01 | 10 | 0.001 | 1, 8, | 2 |
| | | | | | 15, 22 | |
| 2 | IV | 0.1 | 10 | 0.01 | 1, 8, | 2 |
| | | | | | 15, 22 | |
| 3 | IV | 1 | 10 | 0.1 | 1, 8, | 2 |
| | | | | | 15, 22 | |
| 4 | IV | 0.01/1 | 10 | 0.001/0.1 | 1, 2 | 2 |
| 5 | SC | 1 | 1 | 1 | 1 | 2 |
| 6 | SC | 10 | 1 | 10 | 1 | 2 |
| 7 | SC | 20 | 1 | 20 | 36 | 2 |
| 8 | SC | 0.01 | 1 | 0.01 | 57 | 2 |
| 9 | SC | 0.1 | 1 | 0.1 | 57 | 2 |

(Day in table reflects actual study day).

The study was conducted at Charles River Laboratories (Tranent, UK) in accordance with the European Convention for the Protection of Vertebrate Animals Used for Experimental and Other Scientific Purposes (Council of Europe), under control of the UK Home Office.

Purpose-bred cynomolgus monkeys, Macaca fascicularis, of Mauritian origin were obtained from Bioculture (Mauritius) Limited (Riviere de Anguilles, Mauritius) or Noveprim (Mahebourg, Mauritius). Animals were socially housed in gang pens, with environmental enrichment provided.

Sample Collection

Whole blood samples (approximately 0.5 mL) were collected from the femoral vein using sterile hypodermic needles and sterile syringes. For immunophenotyping by flow cytometry, blood was transferred to tubes containing sodium heparin and stored at room temperature until analysis within 48 h.

Biopsies (approximately 20 mg) were taken from superficial lymph nodes by cutting down onto the lymph node using standard surgical aseptic techniques, while the animals were under general anaesthesia. Biopsies were collected in Roswell Park Memorial Institute (RPMI) and stored on wet ice until processing within 24 h. Single cell suspensions were prepared using the Medimachine System for automated, mechanical disaggregation of tissues (Becton Dickinson; see full CRL study reports for details). The resulting cells were re-suspended in 2 mL Dulbecco's phosphate-buffered saline (PBS; Gibco, cat. No. 14190).

At the time of necropsy, samples from lymph nodes and spleen were orientated onto cork discs, individually wrapped in aluminum foil, uniquely labelled, snap frozen in liquid nitrogen and stored in a freezer set to maintain −80° C. pending evaluation by immunohistochemistry.

Flow Cytometry

Mixtures of directly labelled antibodies (see below) were prepared in round bottom test tubes (Falcon, cat. No. 352052). The antibody mixtures were chosen to be able to also analyze CD19+B cells.

For immunophenotyping of peripheral blood, 50 μL of anti-coagulated whole blood was added to the antibody mixture and incubated, protected from light, at RT for 20 min. Red blood cells (RBCs) were lysed using 1×RBC lysis buffer (eBioscience, cat. No. 00-4300-54) at RT for 10 min (or until RBC lysis was complete). The tubes were centrifuged at 300-500 g at RT for 5 min. Supernatant was discarded and the cell pellet re-suspended in 0.5 mL PBS (Gibco, cat. No. 10010). 50 μL of Flow Count beads (Beckman Coulter, cat. No. 7546053) was added to each tube prior to analysis.

For immunophenotyping of lymph node cells, 50 μL of cell suspension was added to the antibody mixture and incubated, protected from light, on ice for 15 min. Following incubation, 0.5 mL Dulbecco's PBS was added to each tube.

The samples were analysed using a two-laser five colour Beckman Coulter FC500 or a BD LSR Fortessa X-20 flow cytometer. CD4-CD8-CD15-CD19+ events were classified as B cells.

The following antibody mixtures were used:

| Antibody¹ | Supplier | Cat. No. |
|---|---|---|
| CD4 FITC | BD Biosciences | 550628 |
| CD16 ECD | Beckman Coulter | A33098/649216* |
| CD8 PC5 | Beckman Coulter | A07758 |
| CD19 PE-Cy7 | Beckman Coulter | IM3628 |

¹FITC: fluorescein isothiocyanate;
PE: phycoerythin;
ECD: Electron coupled dye;
BV: Brilliant violet;
V: violet;
Cy: cyanine dye;
APC: allohphycocyanin
*Supplier changed antibody catalogue number during the study

Immunohistochemistry

Frozen lymph nodes and spleen taken at time of necropsy were sectioned and stained with antibody against CD19 (Abcam, cat. No. ab134114) using immunohistochemistry standard procedures.

Results

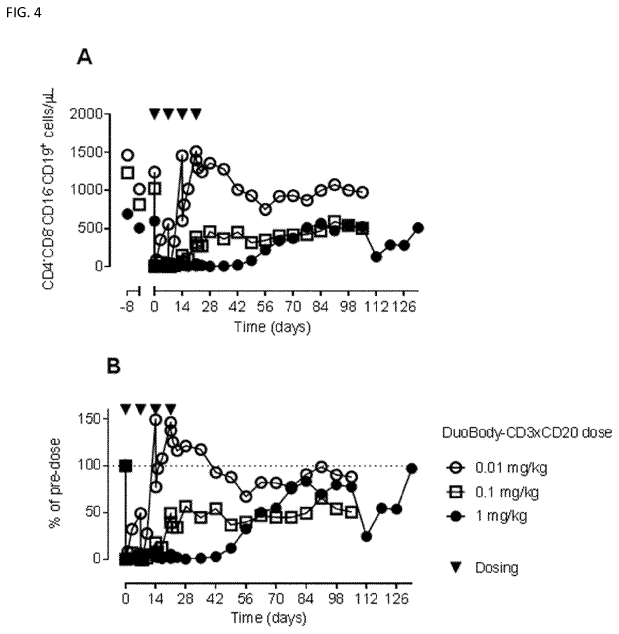

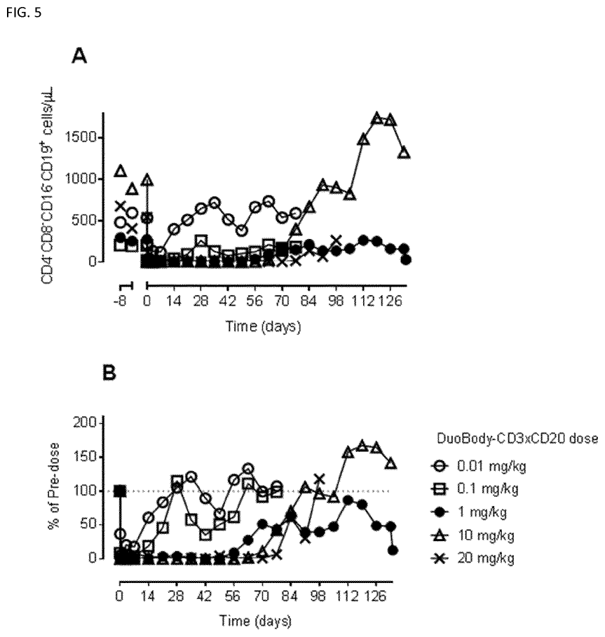

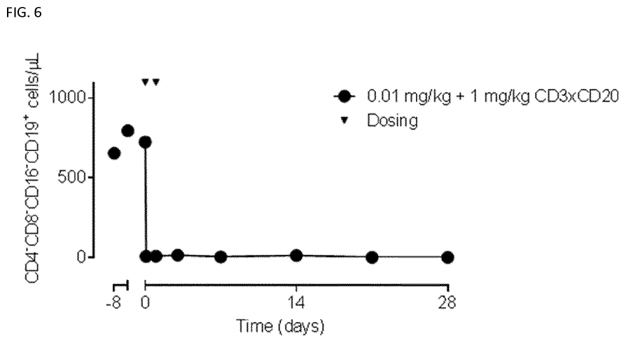

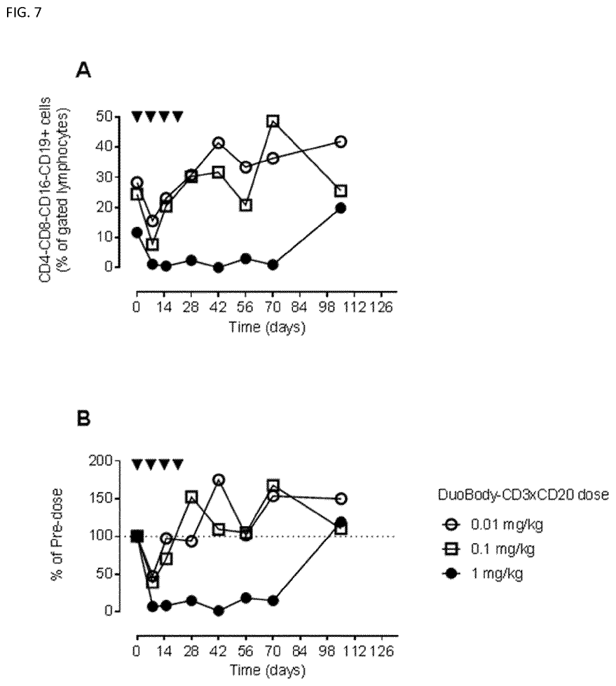

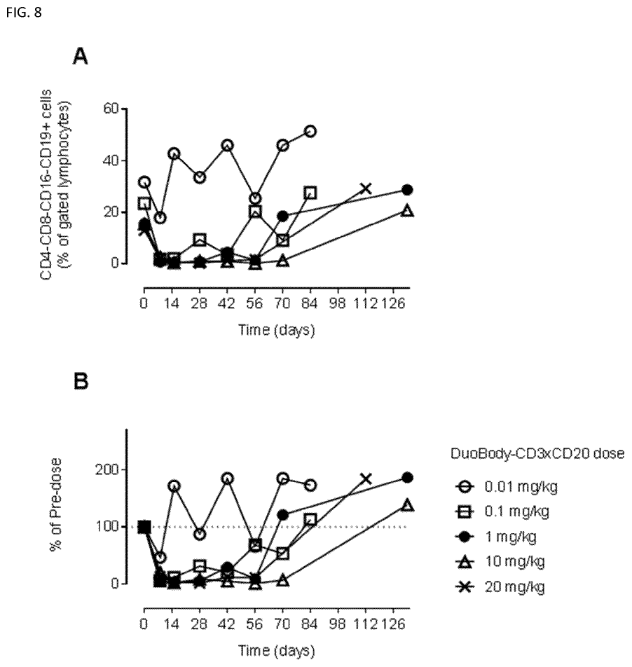

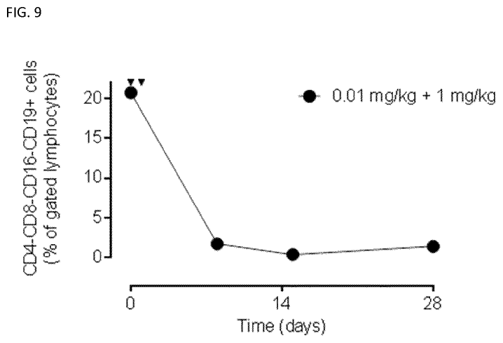

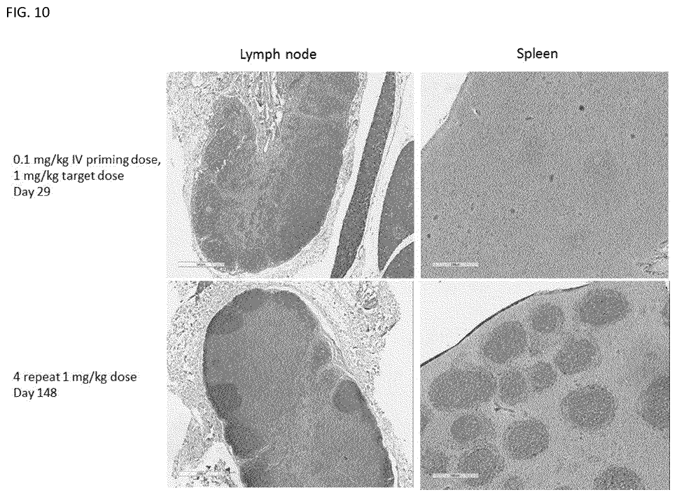

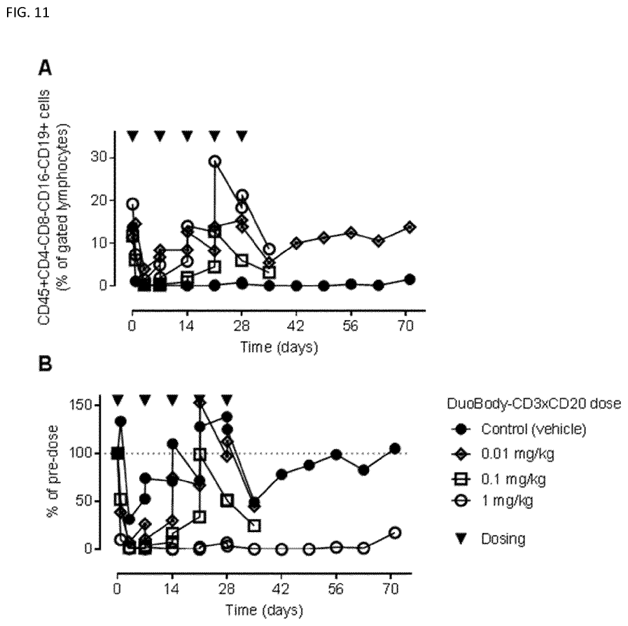

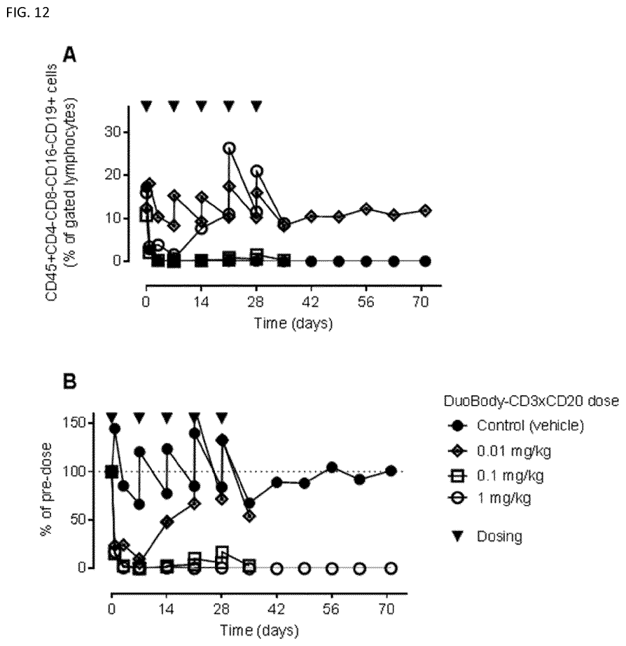

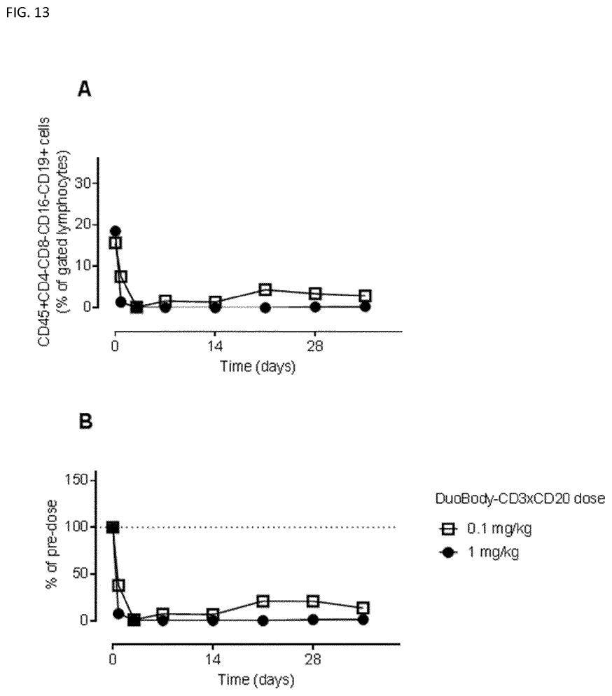

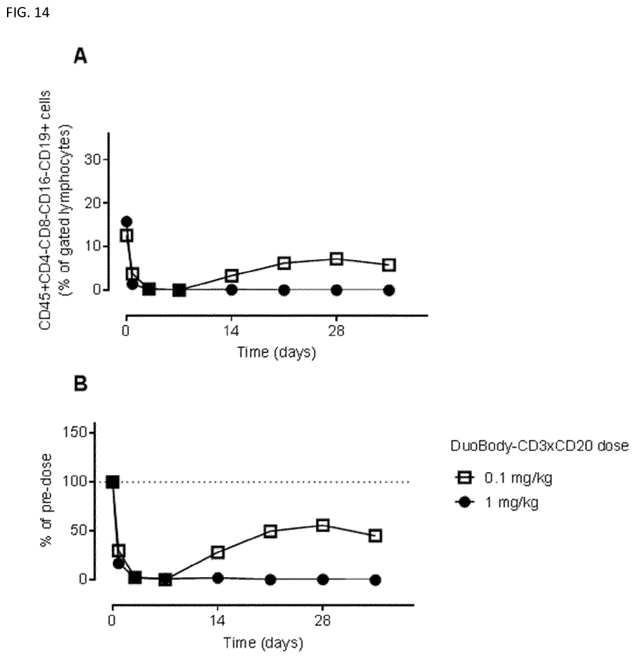

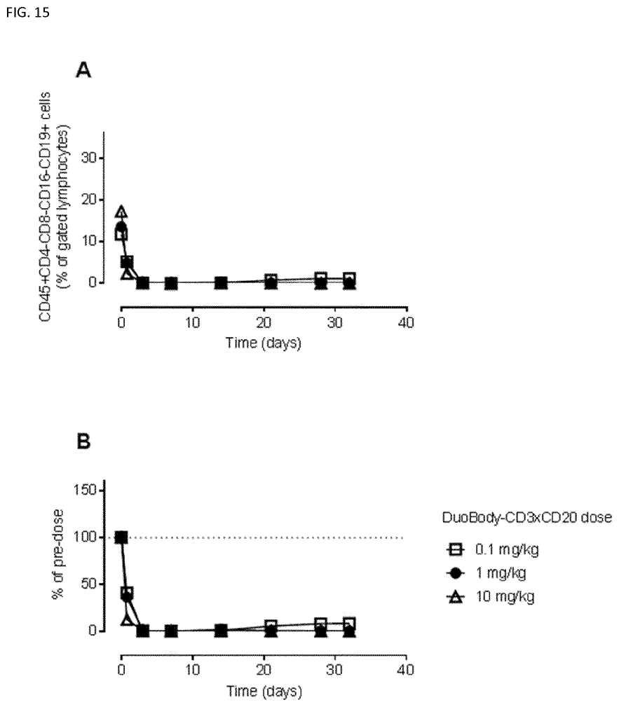

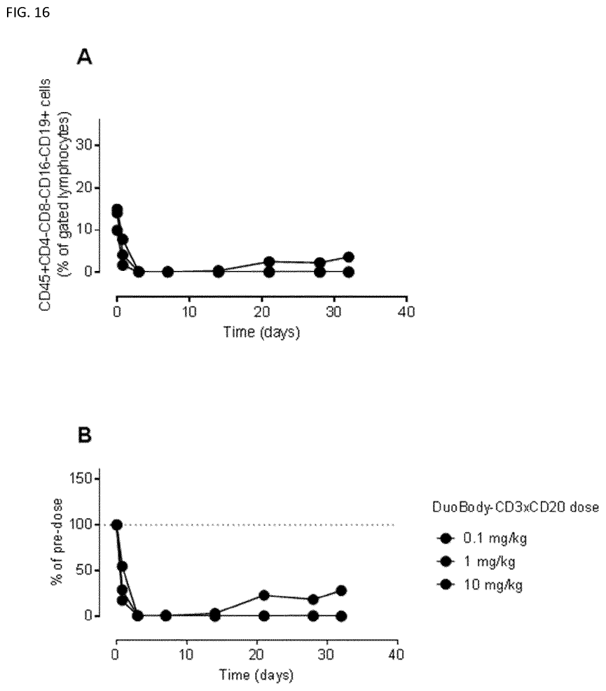

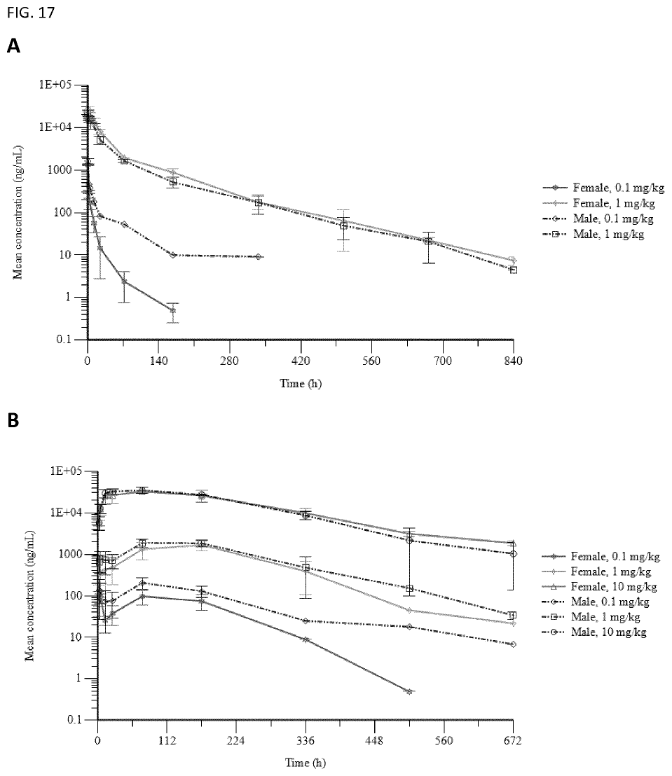

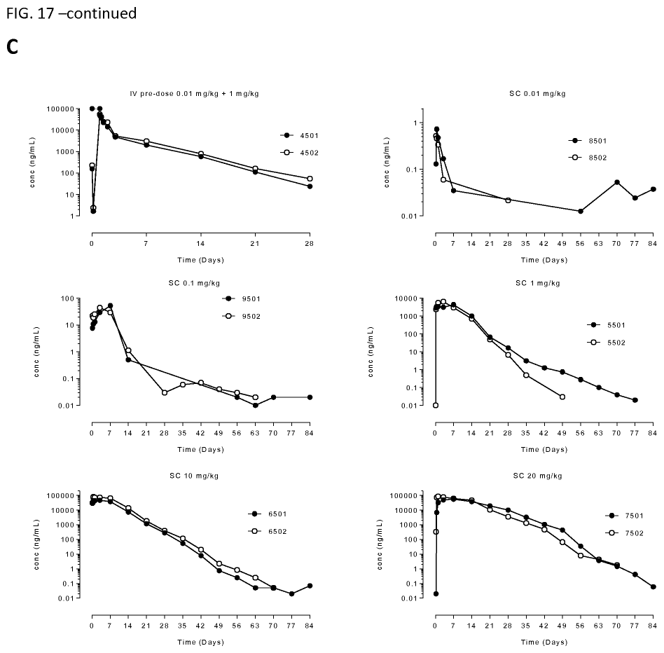

Both repeat IV and single SC administration, as well as IV administration with a priming dose, resulted in dose dependent depletion of B cells from the peripheral blood and lymph nodes (FIGS. 4-9). At 0.01 mg/kg the first two IV doses induced B cell depletion to (nearly) undetectable levels. The 3^{rd} and 4^{th} dose resulted in partial to no B cell depletion. This lack of effect after multiple doses could be due to the formation of anti-drug antibodies. Repeat IV dosing at 0.1 or 1 mg/kg induced complete B cell depletion, with (partial) recovery starting after 21 days (0.1 mg/kg) or after 42-119 days (1 mg/kg). A single SC injection of DuoBody-CD3xCD20 in a pharmaceutical composition of the invention (30 mM acetate, 150 mM sorbitol, pH 5.5) resulted in B cell depletion from the circulation and lymph nodes to undetectable levels at all dose levels. B cell recovery was observed in all groups, returning to baseline in weeks at lower doses, and at around 70 days post-dose at higher doses. IV dosing of a priming dose (0.01 mg/kg) followed by a target dose of 1 mg/kg one day later resulted in complete depletion of B cells from the peripheral blood and lymph nodes, lasting until the day of scheduled necropsy (day 29). In this study, depletion of B cells from lymph nodes and spleen was confirmed by immunohistochemistry (FIG. 10).

### Example 4: B Cell Depletion in Cynomolgus Monkeys Following 5× Repeat Dose IV Infusions or Single Dose SC Injection of DuoBody-CD3xCD20 (GLP Toxicity Study)

Male and female cynomolgus monkeys received DuoBody-CD3xCD20 in a pharmaceutical composition of the invention via 5 weekly IV infusions (0.01, 0.1 or 1 mg/kg), via a single IV infusion (0.1 or 1 mg/kg), or via SC injection (0.1, 1 or 10 mg/kg); a control group receiving 5 weekly IV infusions of saline was also included, as per this overview:

| | | | Dose | Dose | Dose | | | | |
|---|---|---|---|---|---|---|---|---|---|
| | | | level | Volume | Conc | Number of Animals | | | |
| Group | Test | | (mg/ | (mL/ | (mg/ | Main study | | Recovery | |
| No. | Item | Route | kg) | kg) | mL) | Males | Females | Males | Females |
| 1 | Control | IV(1qwx | 0 | 10 | 0 | 3 | 3 | 2 | 2 |
| | (saline) | 5) | | | | | | | |
| 2 | DuoBody | IV(1qwx | 0.01 | 10 | 0.001 | 3 | 3 | — | — |
| | — | 5) | | | | | | | |
| 3 | CD3×CD20 | IV(1qwx | 0.1 | 10 | 0.01 | 3 | 3 | — | — |
| | | 5) | | | | | | | |
| 4 | | IV(1qwx | 1.0 | 10 | 0.1 | 3 | 3 | 2 | 2 |
| | | 5) | | | | | | | |
| 5 | | IV (SD) | 0.1 | 10 | 0.01 | 3 | 3 | — | — |
| 6 | | IV (SD) | 1.0 | 10 | 0.1 | 3 | 3 | — | — |
| 7 | | SC | 0 + 0.1 | 0.2 | 0 + 0.5 | 3 | 3 | — | — |
| | | (2× SD) | | | | | | | |
| 8 | | SC | 0 + 1.0 | 0.2 | 0 + 5 | 3 | 3 | — | — |
| | | (2× SD) | | | | | | | |
| 9 | | SC | 0 + 10 | 0.2 | 0 + 50 | 3 | 3 | — | — |
| | | (2× SD) | | | | | | | |

1qwx5 = Once weekly IV for five occasions (Days 1, 8, 15, 22 and 29); Termination: Day 36 (Main Study); Termination: Day 71 (Recovery).
IV SD = Single IV dose (Day 1); Termination Day 36.
SC 2× SD = SC dose with DuoBody-CD3×CD20 and its SC vehicle (30 nnM acetate buffer, 150 mM sorbitol pH 5.5) on Days 1 and 29, separate injection sites in same animal); Termination Day 33.

The study was conducted at Charles River Laboratories (Tranent, UK) in accordance with the European Convention for the Protection of Vertebrate Animals Used for Experimental and Other Scientific Purposes (Council of Europe), under control of the UK Home Office.

Purpose-bred cynomolgus monkeys, Macaca fascicularis, of Mauritian origin were obtained from LCL-Cynologics (Port-Louis, Mauritius). Animals were socially housed in gang pens, with environmental enrichment provided.

Whole blood samples and lymph node biopsies were collected as described supra.

Quantification of B cells was done by flow cytometry as described supra, except that
for immunophenotyping of peripheral blood, 50 μL of anti-coagulated whole blood was added to the antibody mixture and incubated, protected from light, at +4° C. for 30 min, and
TruCount tubes (BD Biosiences) were used during data acquisition.
CD45+CD4-CD8-CD15-CD19+ events were classified as B cells.
The following antibody mixtures were used:

| Antibody¹ | Supplier | Cat. No. |
|---|---|---|
| CD45 V500/CD45 | BD Biosciences | 561489/740809 |
| BV711* | | |
| CD16 BV650 | Biolegend | 302042 |
| CD4 FITC | BD Biosciences | 550628 |
| CD19 APC | Beckman Coulter | IM2470 |
| CD8 APC-H7 | BD Biosciences | 560179 |

¹FITC: fluorescein isothiocyanate;
PE: phycoerythin;
ECD: Electron coupled dye;
BV: Brilliant violet;
V: violet;
Cy: cyanine dye;
APC: allohphycocyanin
*During this study CD45 V500 was replaced temporarily with CD45 BV711 due to supplier issues. CD45 V500 was reinstated after supplier issues were resolved. As these were detected on different filters the CD25 BV711 was temporarily substituted with CD25 V510.

Results

The results are shown in FIGS. 11-16. IV infusions of saline partially decreased B cell numbers in the peripheral blood, yet B cell numbers returned to baseline within 3 weeks after the last infusion. Five weekly IV infusions of DuoBody-CD3xCD20 induced dose dependent B cell depletion, with partial depletion in the low dose group, and long lasting, complete depletion in the high dose groups. A single IV infusion of 0.1 or 1 mg/kg completely depleted B cells from the peripheral blood, with depletion lasting until day of necropsy (day 36) for the highest dose tested. SC administration of DuoBody-CD3xCD20 resulted in complete B cell depletion from the peripheral blood at all dose levels tested. At the lowest dose B cell levels recovered partially, whereas complete B cell depletion remained until the day of necropsy (day 33) in the 1 and 10 mg/kg groups.

### Example 5: Pharmacokinetics of DuoBody-CD3xCD20 in Cynomolgus Monkeys: Intravenous (IV) and Subcutaneous (SC) Routes of Administration

Materials and Methods

The pharmacokinetic (PK) properties of DuoBody-CD3xCD20 formulated in 30 mM acetate buffer, 150 mM sorbitol and pH 5.5 were determined in cynomolgus monkeys in toxicology studies evaluating both intravenous (IV) and subcutaneous (SC) routes of administration. Blood samples were obtained from animals from a dose-range finding (DRF) study of DuoBody-CD3xCD20 in female cynomolgus monkeys, as well as a GLP toxicology study of DuoBody-CD3xCD20 in cynomolgus monkeys. The designs and details of these studies are described in Example 2. PK evaluations were conducted on the animals that received single IV infusion or SC injection. Blood samples (approximately 0.5 mL each) were obtained for the determination of plasma concentrations of DuoBody-CD3xCD20 from all animals. Concentrations of DuoBody-CD3xCD20 in cynomolgus monkey plasma from the DRF study were determined using an Imperacer® Immuno-PCR assay. Concentrations of DuoBody-CD3xCD20 in cynomolgus monkey plasma from the GLP toxicology study were determined using a single molecule counting (SMC) method. Details of these evaluations are provided in the following sections.

DuoBody-CD3xCD20 Specific PK Imperacer® Immuno-PCR

Concentrations of DuoBody-CD3xCD20 in cynomolgus monkey plasma from the DRF study were determined using the Imperacer® method, an advanced ultra-sensitive immuno-polymerase chain reaction (PCR) technique that utilizes antibody-DNA conjugates and a subsequent exponential amplification of the DNA marker for protein detection. Briefly, an eight-point calibration curve of DuoBody-CD3xCD20 prepared in 100% cynomolgus monkey plasma, quality controls (QCs) and (diluted) cynomolgus monkey test samples were diluted with sample dilution buffer SDB6000 containing Imperacer® conjugate CHI-SAB1 A1 (Chimera Biotec GmbH, Dortmund, Germany, Cat no. 11-272). Samples were added to a 96-well ELISA plate coated with His-tagged extracellular domain of CD3 (CD3ECDHis; Genmab, Utrecht, The Netherlands) to which DuoBody-CD3xCD20 can bind. Plates were washed, PCR mastermix (Molzym, Cat no. C-022) was added and samples were transferred to the Imperacer RT-PCR reader (Enabled RT Cycler MX 3000P/MX 3005P from Agilent Technologies/Chimera). Immobilized DuoBody-CD3xCD20 can be detected during PCR-amplification of the DNA-marker included in the Imperacer® detection conjugate. The processing of a sequence-specific fluorescent probe in the PCR-Mastermix generates an increase of fluorescence signal that is directly related to the amount DNA marker initially present and is reported as ΔCt signal. After completion of realtime PCR signal generation, the measured fluorescence data were processed with instrument software (MXPro; Chimera Biotec GmbH) and analyzed with mathematical software (Microsoft Excel, XLfit analysis plugin). The concentration of bound DuoBody-CD3xCD20 was determined from a standard curve which was made by plotting ΔCt signals against log spiked concentration DuoBody-CD3xCD20 using a non-linear sigmoidal 4-parameter regression. This assay was established and performed at Chimera Biotec GmbH, Dortmund. The LLOQ was 1.0 μg/mL neat plasma.

DuoBody-CD3xCD20 PK Single Molecule Counting (SMC) Method

Concentrations of DuoBody-CD3xCD20 in cynomolgus monkey plasma from the GLP toxicology study were determined using a single molecule counting (SMC) method. The SMC immunoassay is a fluorescent sandwich immunoassay technique that can measure DuoBody-CD3xCD20 molecules in cynomolgus monkey plasma.

In brief, calibrators, QC and study samples were filtered before use and magnetic beads were labelled with an anti-idiotype antibody directed against the CD3 arm of DuoBody-CD3xCD20 (UM-IgG1 mm-3005-101-3-1-MP; Genmab, Utrecht, The Netherlands) according to the manufacturer's protocol (Merck Millipore, Cat no. 03-0077-02). The filtered samples were incubated with the coated magnetic particles w and an anti-idiotype antibody directed against the CD20 arm of DuoBody-CD3xCD20 coupled to fluoroschrome (UM-IgG1 mm-3001-2F2-Sab1.1(-FL); Genmab, Utrecht, The Netherlands). After incubation the particles were washed to remove unbound conjugate. The magnetics particles, with bound analyte and conjugate were then transferred to a clean plate and the remaining buffer was aspirated. The analyte and conjugate were dissociated from the magnetic particles with elution buffer, according to the manufacturer's protocol (Merck Millipore), and the eluate was transferred to a 384-well plate containing neutralization buffer. The samples were drawn into a capillary by the Erenna® single molecule counting system (Merck/Millipore) and illuminated by a laser. The fluorescently labeled molecules emit light and signals above threshold are counted as detected events. In addition, the amount of light of each event (event photons) and the total amount of light (total photons) were measured.

The method was validated and performed at PRA Health Sciences Bioanalytical Laboratory (PRA), Assen, The Netherlands. During the validation, the LLOQ was determined at 0.100 ng/mL neat plasma, and the upper limit of quantification (ULOQ) at 50 ng/mL neat plasma.

Results

Dose Range Finding Study: Single IV Dose with a Priming Dose

Plasma concentration profiles for DuoBody-CD3xCD20 were measured for cynomolgus monkeys that received a priming dose of DuoBody-CD3xCD20 at 0.01 mg/kg on Day 1, followed by a target dose of DuoBody-CD3xCD20 at 1 mg/kg on Day 2 (n=2 females). Both the priming dose and the target dose were administered as an IV infusion at a dose volume of 10 mL/kg over a 30-minute period. Following IV administration of DuoBody-CD3xCD20 as a 1 mg/kg target dose (Day 2) that was preceded by an IV priming dose of 0.01 mg/kg (Day 1), Cₘₐₓ was reached immediately at the end of the infusion of the 1 mg/kg dose. CL values (10.7 to 13.7 mL/day/kg) and V_{D} values (56.1 to 64.9 mL/kg) in the same order of magnitude were observed after the first dose in the multiple dose IV infusion group.

Individual plasma concentration profiles generated from the Imperacer Immuno-PCR method are shown in FIG. 17A and the group mean PK parameters are shown in Table 10. PK parameters were calculated only for the 1 mg/kg dose.

TABLE 10
IV Single Dose: Mean PK Parameters for DuoBody-CD3×CD20 in DRF study

| Dose | | | AUC_{0-∞} | | CL | |
|---|---|---|---|---|---|---|
| (mg/kg) | Tₘₐₓ (day) | Cₘₐₓ (μg/mL) | (ng · day/mL) | T_{1/2} (day) | (mL/day · kg) | V_{D} (mL/kg) |
| 0.01 + 1 | 0 | 51.6 | 83259 | 3.46 | 12.2 | 60.5 |

Dose Range Finding Study: Single SC Dose

Plasma concentration profiles for DuoBody-CD3xCD20 were measured after SC single dose injection of DuoBody-CD3xCD20 at dose levels of 0.01, 0.1, 1, 10, or 20 mg/kg (n=2 females/group). SC injections were administered at a dose volume of 1 mL/kg. Following SC administration, Cₘₐₓ was reached between 0.5 and 7 days after dosing. Based on either nCₘₐₓ or AUC_{0-∞}, a super-proportional increase in exposure was observed up to a dose of 1 mg/kg. Between 1 mg/kg and 20 mg/kg, a proportional increase in exposure was observed with increasing dose. Individual plasma concentration profiles generated from the Imperacer® Immuno-PCR method are shown in FIG. 17 B and group mean PK parameters are shown in Table 11.

The absolute SC bioavailability (F) was calculated as a percentage of the IV bioavailability using the AUC_{inf} after a SC administration of 1 mg/kg and the AUC_{0-∞} after the first IV dose of 1 mg/kg, and was found to be 111%, indicating a complete (100%) SC bioavailability at this dose.

TABLE 11
SC Single Dose: Mean PK Parameters for DuoBody-CD3×CD20 in DRF study

| | | | nCmax | | | |
|---|---|---|---|---|---|---|
| Dose | Tₘₐₓ | Cₘₐₓ | (μg/mL)/ | T_{1/2} | AUC_{0-∞} | |
| (mg/kg) | (day) | (μg/mL) | (mg/kg) | (day) | (ng · h/mL) | nAUC_{0-∞} |
| 0.01 | 0.334 | 0.00063 | 0.063 | 31.7 (n = 1) | 4.67 (n = 1) | ng · day/mL)/ |
| 0.1 | 5 | 0.049 | 0.49 | 12.1 | 385 | (mg/kg) |
| 1 | 5 | 5.43 | 5.43 | 3.68 | 49004 | 467.3 |
| | | | | | | (n = 1) |
| 10 | 1.75 | 62.1 | 6.21 | 3.34 | 653611 | 3848 |
| 20 | 1.75 | 69.2 | 3.45 | 3.81 | 693552 | 49004 |

GLP Toxicity Study: Single IV Dose

Plasma concentration profiles for DuoBody-CD3xCD20 were measured after a single dose IV infusion of DuoBody-CD3xCD20 at dose levels of 0.1 or 1 mg/kg (3 monkeys/sex/group). Group mean plasma concentration profiles generated from the SMC method are shown in FIG. 17 C and group mean pharmacokinetic parameters are shown in Table 12. Systemic exposure to DuoBody-CD3xCD20 (based on mean Cₘₐₓ and AUC₍₀₋ₜ₎ increased with increasing dose in males and females. Based on dose-normalized estimates, systemic exposure to DuoBody-CD3xCD20 increased in a generally greater than dose-proportional manner between 0.1 and 1 mg/kg dose range in both, males and females. Median Tₘₐₓ was consistently 0.5 hours (end of infusion period). A trend was noted for CL, VD and VSS to decrease as the dose increased. T_{1/2} was likely to be appropriately derived at the higher dose of 1 mg/kg (mean values of 98 and 125 hours in males and females, respectively) where the elimination phase was characterized up to 840 hours in both sexes compared to 168 or 336 hours at 0.1 mg/kg. Systemic exposure was generally comparable between males and females at 0.1 and 1 mg/kg, although mean AUC₍₀₋ₜ₎ in males was greater than females dosed at 0.1 mg/kg. This is likely an artefact of one animal that exhibited a total exposure that was approximately 7-fold greater than that in all other animals. When accounting for variability due to this animal, female/male ratios of Cₘₐₓ and AUC₍₀₋ₜ₎ ranged from 0.8 to 1.3 (0.3 for AUC₍₀₋ₜ₎ at 0.1 mg/kg).

TABLE 12
IV Single Dose: Mean PK Parameters for DuoBody-CD3×CD20 in GLP
toxicology study

| Dose | | Cₘₐₓ | AUC_{0-∞} | | | |
|---|---|---|---|---|---|---|
| (mg/kg) | Tₘₐₓ (h) | (ng/mL) | (ng· h/mL) | T_{1/2} (h) | CL (mL/h · kg) | V_{D} (mL/kg) |
| 0.1 | 0.5 | 1610 (M) | 13500 (M) | 47.5 (M) | 21.6 (M) | 1060 (M) |
| | | 1300 (F) | 4280 (F) | 32.7 (F) | 24.1 (F) | 1160 (F) |
| 1 | 0.5 | 21600 (M) | 615000 (M) | 98.4 (M) | 1.63 (M) | 233 (M) |
| | | 28400 (F) | | 125 (F) | 1.21 (F) | 226 (F) |

840000 (F)

Single SC Dose

Plasma concentration profiles for DuoBody-CD3xCD20 were measured after a single dose SC injection of DuoBody-CD3xCD20 at dose levels of 0.1, 1, or 10 mg/kg (3 monkeys/sex/group). SC injections were administered at a dose volume of 0.2 mL/kg. Group mean plasma concentration profiles generated from the SMC method are shown in FIG. 17C and group mean pharmacokinetic parameters are shown in Table 13. Systemic exposure to DuoBody-CD3xCD20 (based on mean Cₘₐₓ and AUC₍₀₋ₜ₎) increased with increasing SC dosing in males and females. Based on dose-normalized estimates, Cₘₐₓ increased in a generally dose-proportional manner between 0.1 and 1 mg/kg and greater than dose-proportionally between 1 and 10 mg/kg in males and females, whereas dose-normalized AUC₍₀₋ₜ₎ increased greater than dose-proportionally from 0.1 to 10 mg/kg. Overall, the increase was greater than dose-proportional from 0.1 to 10 mg/kg in males and females after SC dosing. Median Tₘₐₓ was consistently 72 hours in males with no consistent trends in Tₘₐₓ noted in females across the dose range, due to greater variability across individual Tₘₐₓ values. T₁ was longest at the high dose where the elimination phase appeared to be most appropriately characterized in males and females. Systemic exposure was generally greater in males than females at 0.1 mg/kg and comparable between males and females at 1 and 10 mg/kg; female/male ratios of Cₘₐₓ and AUC₍₀₋ₜ₎ were 0.5 and 0.4, respectively, at 0.1 mg/kg, 0.8 for both parameters at 1 mg/kg and 1.0 for both parameters at 10 mg/kg.

TABLE 13
SC Single Dose: Mean PK Parameters for DuoBody-CD3xCD20 in GLP
toxicology study

| Dose | Tₘₐₓ | Cₘₐₓ | AUC_{0-∞} | T_{1/2} | F |
|---|---|---|---|---|---|
| (mg/kg) | (h) | (ng/mL) | (ng · h/mL) | (h) | (%) |
| 0.1 | 72 | 244 (M) | 48300 (M) | 86.4 (M) | 358 (M) |
| | 4 | 111 (F) | 27400 (F) | 43.0 (F) | 640 (F) |
| 1 | 72 | 2100 (M) | 520000 (M) | 62.1 (F) | 85 (M) |
| | 168 | 1670 (F) | 412000 (F) | 55.2 | 49 (F) |
| 10 | 72 | 35900 (M) | 9540000 (M) | 102 (M) | — |
| | 72 | 35100 (F) | 9620000 (F) | 145 (F) | — |

Taken together, following IV infusion of DuoBody-CD3xCD20, plasma concentrations increased up to the end of the 30-minute dosing period to then decrease in a generally bi-phasic manner. After SC dosing, a more prolonged increase was observed up to a peak approximately 72 hours post dose and remained at a relatively steady level up to 168 hours post dose. Thereafter, concentrations decreased in a mono-phasic manner up to the end of the 4-week sampling period. At equivalent doses, the maximal plasma concentration after IV dosing was significantly higher than the maximal plasma concentration after SC dosing.

1. A pharmaceutical composition comprising or consisting essentially of:
a. 50 to 120 mg/mL of a bispecific antibody binding to human CD3 and human CD20,
b. 20 to 40 mM acetate
c. 140 to 160 mM sorbitol
where the pH of the composition is from 5 to 6 and where the bispecific antibody comprises a first binding region binding to human CD3 which comprises the CDR sequences:
VH-CDR1: SEQ ID NO:1
VH-CDR2: SEQ ID NO:2
VH-CDR3: SEQ ID NO: 3
VL-CDR1: SEQ ID NO: 4
VL-CDR2: GTN, and
VL-CDR3: SEQ ID NO: 5
and a second binding region binding to human CD20 which comprises the CDR sequences:
VH-CDR1: SEQ ID NO: 8
VH-CDR2: SEQ ID NO: 9
VH-CDR3: SEQ ID NO:10
VL-CDR1: SEQ ID NO: 11
VL-CDR2: DAS, and
VL-CDR3: SEQ ID NO: 12.

2. The pharmaceutical composition of claim 1 wherein the first binding region of the bispecific antibody binding to CD3 comprises a VH and a VL sequence having at least 90% sequence identity to the VH and VL sequences of SEQ ID: 6 and 7, such as at least 95%, 96%, 97%, 98%, 99% or 100% sequence identity to the VH and VL sequences of SEQ ID: 6 and 7.

3. The pharmaceutical composition of claim 1 or 2 wherein the second binding region of the bispecific antibody binding to CD20 comprises a VH and a VL sequence having at least 90% sequence identity to the VH and VL sequences of SEQ ID: 13 and 14, such as at least 95%, 96%, 97%, 98%, 99% or 100% sequence identity to the VH and VL sequences of SEQ ID: 13 and 14.

4. The pharmaceutical composition of any of claims 1-3 wherein the bispecific antibody is an IgG1 antibody.

5. The pharmaceutical composition of any one of claims 1-4 wherein the bispecific antibody comprises a first and a second light chain which comprises a first and a second light chain constant region which is selected between a lambda light chain constant region and a kappa light chain constant region such as the light chain constant regions of SEQ ID Nos 22 and 23.

6. The pharmaceutical composition of any one of the above claims wherein the bispecific antibody comprises an Fc region which comprises a first and second heavy chain, wherein said Fc region has been modified so that it has reduced effector functions compared to the bispecific antibody comprising a wild-type IgG1 Fc region.

7. The pharmaceutical composition of any one of the above claims wherein the bispecific antibody comprises an Fc region which has been modified so that binding of C1q to said antibody is reduced compared to the bispecific antibody having a wild-type IgG1 Fc region by at least 70%, at least 80%, at least 90%, at least 95%, at least 97%, or 100%, wherein C1q binding is determined by ELISA.

8. The pharmaceutical composition of any one of the above claims wherein the bispecific antibody comprises a first and second heavy chain each comprising at least a hinge region, a CH2 and CH3 region, wherein in said first heavy chain at least one of the amino acids in the positions corresponding to a positions selected from the group consisting of T366, L368, K370, D399, F405, Y407, and K409 in a human IgG1 heavy chain has been substituted, and in said second heavy chain at least one of the amino acids in the positions corresponding to a position selected from the group consisting of T366, L368, K370, D399, F405, Y407, and K409 in a human IgG1 heavy chain has been substituted, and wherein said first and said second heavy chains are not substituted in the same positions.

9. The pharmaceutical composition of any one of the above claims wherein (i) the amino acid in the position corresponding to F405 in a human IgG1 heavy chain is L in said first heavy chain, and the amino acid in the position corresponding to K409 in a human IgG1 heavy chain is R in said second heavy chain, or (ii) the amino acid in the position corresponding to K409 in a human IgG1 heavy chain is R in said first heavy chain, and the amino acid in the position corresponding to F405 in a human IgG1 heavy chain is L in said second heavy chain.

10. The pharmaceutical composition of any one of the above claims wherein the positions corresponding to positions L234 and L235 in the human IgG1 heavy chain of both the first heavy chain and the second heavy chain of the bispecific antibody are F and E, respectively.

11. The pharmaceutical composition of any one of the above claims wherein the positions corresponding to positions L234, L235, and D265 in the human IgG1 heavy chain of both the first heavy chain and the second heavy chain of the bispecific antibody are F, E, and A, respectively.

12. The pharmaceutical composition of any one of the above claims wherein the positions corresponding to positions L234, L235, and D265 in the human IgG1 heavy chain of both the first constant heavy chain and the second constant heavy chain of the bispecific antibody are F, E, and A, respectively, and wherein the position corresponding to F405 in the human IgG1 heavy chain of the first constant heavy chain is L, and the position corresponding to K409 in the human IgG1 heavy chain of the second constant heavy chain is R.

13. The pharmaceutical composition of any one of the above claims wherein the first and second constant heavy chains comprises an amino acid sequence having at least 90% identity to the amino acid sequence of SEQ ID NO:16.

14. The pharmaceutical composition of any one of the above claims wherein the first and second constant heavy chains comprise the amino acid sequence of SEQ ID Nos: 19 and 20, respectively.

15. The pharmaceutical composition of any one of the above claims wherein a. is 50 to 120 mg/mL such as 50 to 110 mg/mL, or such as 50 to 100 mg/mL, such as 50 to 90 mg/mL, such as 50 to 80 mg/mL, such as 50 to 70 mg/mL, such as 55 to 65 mg/ml, such as 58 to 62 mg/ml, such as 60 mg/mL or a is about 120 mg/mL.

16. The pharmaceutical composition of any one of the above claims wherein b. is 28 to 32 mM such as 30 mM.

17. The pharmaceutical composition of any one of the above claims wherein c. is 145 to 155 mM such as 148 to 152 mM such as 150 mM.

18. The pharmaceutical composition of any one of the above claims wherein pH is 5.3 to 5.6, such as 5.4 to 5.6 such as about 5.5.

19. The pharmaceutical composition of any one of the above claims wherein the composition has a pH of 5.4 to 5.6, such as 5.5 and consists essentially of:
a. 50 to 120 mg/mL of the bispecific antibody
b. 20 to 40 mM acetate
c. 140 to 160 mM sorbitol.

20. The pharmaceutical composition of any one of the above claims wherein the composition has a pH of 5.4 to 5.6 and consists essentially of:
a. 58 to 62 mg/mL of the bispecific antibody
b. 28 to 32 mM acetate
c. 145 to 155 mM sorbitol

21. The pharmaceutical composition of any one of the above claims wherein the composition has a pH of 5.5 and consists essentially of:
a. 60 mg/mL of the bispecific antibody
b. 30 mM acetate
c. 150 mM sorbitol

22. The pharmaceutical composition of any one of the above claims 1 to 19 wherein the composition has a pH of 5.4 to 5.6 and consists essentially of:
a. 110 to 130 mg/mL of the bispecific antibody
b. 28 to 32 mM acetate
c. 145 to 155 mM sorbitol

23. The pharmaceutical composition of any one of the above claim 22 wherein the composition has a pH of 5.5 and consists essentially of:
a. 120 mg/mL of the bispecific antibody
b. 30 mM acetate
c. 150 mM sorbitol

24. The pharmaceutical composition of any one of the above claims wherein the composition does not comprise a surfactant.

25. The pharmaceutical composition of any one of the above claims wherein the composition does not comprise a hyaluronidase.

26. The pharmaceutical composition of any one of the above claims wherein the composition is a subcutaneous composition.

27. The pharmaceutical composition of any one of the above claims wherein the composition is an intravenous composition.

28. The pharmaceutical composition of any one of the above claims wherein the composition is for use in the treatment of cancer.

29. The pharmaceutical composition of any one of the above claims wherein the composition is for use in subcutaneous administration.

30. The pharmaceutical composition of any one of the above claims 1-24 wherein the composition is for use in intravenous administration.

31. The pharmaceutical composition of any one of the above claims 1-30 which is in a dosage unit form.

32. The pharmaceutical composition of any one of the above claims which composition is stable for pharmaceutical use for at least 6 months, such as at least 9 month or at least 12 months at a storage temperature of 2-8° C., such as 5° C.

33. Use of the pharmaceutical composition of any one of claims 1-25 for subcutaneous administration.

34. Use of the pharmaceutical composition of any one of claims 1-25 for intravenous administration.

35. The use of any one of claim 33 or 34 wherein the use is for the treatment of cancer.

36. A method of treating cancer in a subject comprising administering to a subject in need thereof the pharmaceutical composition of any one of claims 1 to 32 for a time sufficient to treat the cancer.

37. The method of claim 36 wherein the composition is administered subcutaneously or intravenously.

38. The method of any one of claim 36 or 37 wherein the cancer is a B-cell malignancy.

39. A unit dosage form, comprising or consisting essentially of
a. a bispecific antibody comprising a first binding region binding to human CD3 which comprises the CDR sequences:
VH-CDR1: SEQ ID NO: 1
VH-CDR2: SEQ ID NO: 2
VH-CDR3: SEQ ID NO:3
VL-CDR1: SEQ ID NO: 4
VL-CDR2: GTN, and
VL-CDR3: SEQ ID NO: 5,
and a second binding region binding to human CD20 which comprises the CDR sequences:
VH-CDR1: SEQ ID NO: 8
VH-CDR2: SEQ ID NO: 9
VH-CDR3: SEQ ID NO: 10
VL-CDR1: SEQ ID NO: 11
VL-CDR2: DAS, and
VL-CDR3: SEQ ID NO: 12
in an amount of from 5 μg to 50 mg,
b. acetate buffer and sorbitol in a ratio of between 1:5 and 1:10 wherein the osmolality of the unit dosage form is from 210 to 250 and the pH is from 5.4 to 5.6.

40. A unit dosage form, comprising or consisting essentially of:
a. a bispecific antibody comprising a first binding region binding to human CD3 which comprises the CDR sequences:
VH-CDR1: SEQ ID NO: 1
VH-CDR2: SEQ ID NO:2
VH-CDR3: SEQ ID NO: 3
VL-CDR1: SEQ ID NO: 4
VL-CDR2: GTN, and
VL-CDR3: SEQ ID NO: 5,
and a second binding region binding to human CD20 which comprises the CDR sequences:
VH-CDR1: SEQ ID NO: 8
VH-CDR2: SEQ ID NO: 9
VH-CDR3: SEQ ID NO: 10
VL-CDR1: SEQ ID NO: 11
VL-CDR2: DAS, and
VL-CDR3: SEQ ID NO: 12
in an amount of from 5 μg to 50 mg,
b. acetate at a concentration of 30 mM,
c. sorbitol at a concentration of 150 mM,
at a pH of 5.5.

41. The unit dosage form of any one of claim 39 or 40 wherein the first binding region of the bispecific antibody binding to human CD3 comprises the VH and VL sequences of SEQ ID: 6 and 7 and the second binding region of the bispecific antibody binding to human CD20 comprises the VH and VL sequences of SEQ ID: 13 and 14.

42. The unit dosage form of claim 41 wherein the bispecific antibody comprises the first and second constant region heavy chains of SEQ ID NOs: 19 and 20 respectively.

43. The unit dosage form of any one of claims 39 to 42 wherein the amount of the bispecific antibody is from 50 μg to 40 mg.

44. The unit dosage form of any one of claims 39 to 43 wherein the amount of the bispecific antibody is from 100 μg to 30 mg, such as 150 μg, 200 μg, 250 μg, 300 μg, 350 μg, 400 μg, 450 μg, 500 μg, 600 μg, 700 μg, 800 μg, 900 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg such as 30 mg.

45. The unit dosage form of any one of claim 39 to 44 wherein the total volume is from 0.5 mL to 2 mL, such as 1 mL.

46. The unit dosage form of claim 45 which unit dosage form is for subcutaneous administration.

47. The unit dosage form of any one of claim 39 to 44 wherein the total volume is from 20 mL to 200 mL wherein the dosage form is for I.V. administration.

48. A method of treating cancer in a subject comprising administering to a subject in need thereof the unit dosage form of any one of claims 39 to 47 for a time sufficient to treat the cancer.

49. The unit dosage form of any one of claims 39 to 47 for use in the treatment of cancer.

50. A container comprising the unit dosage form of any one of claims 39 to 45.

51. A kit-of-parts comprising:
a. the pharmaceutical composition of any one of claims 1 to 25
b. a diluent comprising acetate and sorbitol
c. a receptacle for the unit dosage form
d. directions for dilution and/or for use.

52. The kit-of-parts of claim 51 wherein the ratio of the concentrations of acetate and sorbitol is identical in the diluent and the pharmaceutical composition.

53. The kit-of-parts of any one of claim 51 or 52 wherein
a. the pharmaceutical composition comprises:
i. 60 mg/mL of the bispecific antibody
ii. 30 mM acetate buffer
iii. 150 mM sorbitol
iv. pH is 5.5
b. the diluent comprises:
i. 30 mM acetate buffer
ii. 150 mM sorbitol
c. a receptacle for the unit dosage form, and
d. directions for dilution and/or for use.

54. A method of preparing a pharmaceutical composition as defined in any one of claims 1 to 32, comprising the steps of mixing in water for injection:
a. 60 to 120 mg/mL of a bispecific antibody comprising a first binding region binding to human CD3 which comprises the CDR sequences:
VH-CDR1: SEQ ID NO: 1
VH-CDR2: SEQ ID NO: 2
VH-CDR3: SEQ ID NO: 3
VL-CDR1: SEQ ID NO: 4
VL-CDR2: GTN, and
VL-CDR3: SEQ ID NO: 5,
and a second binding region binding to human CD20 which comprises the CDR sequences:
VH-CDR1: SEQ ID NO:8
VH-CDR2: SEQ ID NO: 9
VH-CDR3: SEQ ID NO: 10
VL-CDR1: SEQ ID NO: 11
VL-CDR2: DAS, and
VL-CDR3: SEQ ID NO: 12
b. 3.53 mg/mL of sodium acetate trihydrate
c. 0.32 mg/mL of acetic acid
d. 27.3 mg/mL of sorbitol
and adjusting the pH to 5.5 by adding sodium hydroxide.

55. The method of claim 54 wherein a. is 60 mg/mL.

56. The method of claim 54 wherein a. is 120 mg/mL.

57. A method of preparing a unit dosage form as defined in any one of claims 39 to 45, comprising the steps of:
a. preparing the pharmaceutical composition by the method of any of claims 54 to 56
b. preparing a diluent in water for injection comprising:
i. 3.53 mg/mL of sodium acetate trihydrate
ii. 0.32 mg/mL of acetic acid
iii. 27.3 mg/mL of sorbitol
iv. sodium hydroxide to adjust pH to 5.5
c. mixing the pharmaceutical composition and the diluent to a desired bispecific antibody concentration.

58. A pharmaceutical composition or a unit dosage form, which is obtainable by the method as defined in any one of claims 54 to 57.


SEQUENCE LISTING (25 sequences)

SEQ ID NO: 1 - protein, 8 aa, mus musculus
GFTFNTYA

SEQ ID NO: 2 - protein, 10 aa, mus musculus
IRSKYNNYAT

SEQ ID NO: 3 - protein, 16 aa, mus musculus
VRHGNFGNSYVSWFAY

SEQ ID NO: 4 - protein, 9 aa, mus musculus
TGAVTTSNY

SEQ ID NO: 5 - protein, 9 aa, mus musculus
ALWYSNLWV

SEQ ID NO: 6 - protein, 125 aa, artificial
  humanized variable antibody region
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 7 - protein, 109 aa, artificial
  humanized antibody variable region
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 8 - protein, 8 aa, Homo Sapiens
GFTFHDYA

SEQ ID NO: 9 - protein, 8 aa, Homo Sapiens
ISWNSGTI

SEQ ID NO: 10 - protein, 15 aa, Homo Sapiens
AKDIQYGNYYYGMDV

SEQ ID NO: 11 - protein, 6 aa, Homo Sapiens
QSVSSY

SEQ ID NO: 12 - protein, 9 aa, Homo Sapiens
QQRSNWPIT

SEQ ID NO: 13 - protein, 122 aa, Homo Sapiens
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 14 - protein, 107 aa, Homo Sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 15 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 330 aa, artificial
  modified antibody heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 330 aa, artificial
  modified antibody heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 330 aa, artificial
  modified antibody heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 19 - protein, 330 aa, artificial
  modified antibody heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 20 - protein, 330 aa, artificial
  modified antibody heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 21 - protein, 107 aa, Homo Sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 107 aa, Homo sapiens [sequence omitted]

SEQ ID NO: 24 - protein, 215 aa, Artificial Sequence
  humanized antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 214 aa, artificial
  humanized antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC